(19) United States
(12) Patent Application Publication
(10) Pub. No.: US 2026/0306420 A1
(43) Pub. Date: Oct. 8, 2026

(54) STABLE POLYMER-LIPID NANOPARTICLE FOR DRUG BINDING AND DELIVERY, ESPECIALLY WITH ANTICANCER PROPERTIES, AND A METHOD FOR PRODUCING STABLE POLYMER-LIPID NANOPARTICLES CARRYING DRUGS, ESPECIALLY WITH ANTICANCER PROPERTIES

(71) Applicant: BS BIOTECHNA SPOLKA AKCYJNA, Krakow (PL)

(72) Inventors: Olga SWIECH, Bieniewice (PL); Rafal MACKIEWICZ, Wyszegrod (PL); Weronika PIOTROWSKA, Warszawa (PL); Natalia LABEDZ, Lodz (PL); Mateusz PSURSKI, Wroclaw (PL); Joanna WIETRZYK, Wroclaw (PL); Wojciech SMORON, Krakow (PL); Julia WITEK, Sieradz (PL)

(21) Appl. No.: 19/480,960

(22) PCT Filed: May 3, 2024
(86) PCT No.: PCT/PL2024/000023

§ 371 Date: Nov. 3, 2025

(30) Foreign Application Priority Data
May 3, 2023 (PL) P.444724

(51) Int. Cl. A61K 9/51; A61K 9/1277; A61K 31/395; A61K 31/553

(52) U.S. Cl. CPC A61K 9/5153; A61K 9/1277; A61K 9/5123; A61K 9/5138; A61K 9/5192; A61K 31/395; A61K 31/553

(57) ABSTRACT

A stable polymer-lipid nanoparticle for drug binding and delivery comprising a core containing polylactic-co-glycolic acid (PLGA) and polyvinyl alcohol (PVA) and a core envelope containing a mixture of lipids: 1,2-dipalmitoyl-sn-glycero-3-phosphocholine (DDPC), cholesterol and the ammonium salt of 1,2-distearoyl-sn-glycero-3-phospho-ethanolamine-N-[arnino(polyethylene glycol)-2000 (DSPE-PEG(2000)NH2] and a method for producing such particles.

The object of the invention is a stable polymer-lipid nanoparticle for the binding and delivery of drugs, in particular with anticancer properties, and a method for producing stable polymer-lipid nanoparticles carrying drugs, in particular with anticancer properties.

Polylactic-co-glycolic acid (PLGA) is an FDA-approved copolymer of lactic acid and glycolic acid. It is also one of the most widely used polymers in the design and formulation of drug delivery systems for biomedical applications due to its: biodegradability, biosafety, biocompatibility, formulation versatility and functionalisation. PLGA-based nanoparticles provide: optimal bioavailability of the encapsulated drug, protecting it from premature degradation in the biological environment, regulated degradation kinetics and targeted drug delivery. In addition, they facilitate the intracellular penetration of the bioactive compound and reduce the number of side effects of the therapy. PLGA nanoparticles have been widely used in the following therapies: anticancer [DOI: 10.1615/CritRevTherDrugCarrierSyst.v21.i5.20], anti-inflammatory [https://doi.org/10.1039/C5RA17686G], cardiovascular and immunological [https://doi.org/10.1016/j.drudis.2014.09.018, https://doi.org/10.1039/C5TB00434A].

The use of PLGA allows the encapsulation of a wide range of bioactive molecules including small drugs, proteins and nucleic acids. Due to their low intrinsic toxicity and easy biodegradability, PLGA-containing nanoparticles can be used in formulations for systemic (parenteral), oral and inhalation administration.

Nevertheless, in addition to the attractive features of PLGA-based nanoparticles, there are some limitations usually related to their physicochemical and biological properties, among which we can mention: poor drug saturation (especially in the case of lipophilic therapeutic agents), pharmaceutical ejection in the first hours of administration, phagocytic uptake, short half-life, aggregation, immune responses and uncontrolled distribution in tissues [https://doi.org/10.1016/j.drudis.2014.09.018, https://doi.org/10.1080/17425247.2016.1182492], These properties limit the use of PLGA nanoparticles in biomedicine, so hybrid nanoparticles are being sought in which PLGA is combined with a protective material mainly based on polymers, surfactants and lipids.

The polymer PVA, or poly(vinyl alcohol), is the emulsifier most commonly used to stabilise emulsions during the formation of PLGA nanoparticles because it forms particles with relatively small sizes and a uniform size distribution. The PVA coating remains bonded to the nanoparticles despite repeated washing because PVA forms an interconnected network with the polymer at the interface [https://doi.org/10.1016/0168-3659(95)00070-0]. The PVA coating alters the properties of the PLGA nanoparticles and may reduce the intracellular uptake of nanoparticles which is related to the increased hydrophilicity of the nanoparticle surface and the negative charge on the nanoparticle surface.

PLGA-lipid hybrid nanoparticles have become a more promising approach in modern nanomedicine, improving the pharmacokinetics and biodistribution of the therapeutic system in biological environments while minimising side effects. PLGA-lipids combine the features of biomimetic lipids with the architectural advantages of PLGA nanoparticles into a single entity [https://doi.org/10.3109/21691401.2014.951721, https://doi.org/10.1517/17425247.2016.1151872]. Among polymer-lipid materials, we distinguish between lipid core-polymer envelope or polymer core-lipid envelope nanoparticles. The second type of hybrid nanostructure consists of a biodegradable PLGA core that surrounds the therapeutic agents and which is embedded in a lipid or lipid envelope containing a polyethylene glycol (PEG) modification [https://doi.org/10.2147/IJN.S40579, https://doi.org/10.1039/C2NR32880A, https://doi.org/10.1016/j.ejpb.2013.07.002]. The outer lipid layer reduces the degradation rate of the PLGA core, limiting the diffusion of water within the molecule, allowing for more controlled release kinetics. In addition, this layer acts as a molecular barrier minimising drug leakage at the preparation or storage stage [https://doi.org/10.1039/C2NR32880A, https://doi.org/10.1016/j.ejpb.2013.07.002]. The outer layer of the carrier is also responsible for the circulation time of the carrier in the bloodstream and its accumulation in specific organs and tissues. The composition of the outer lipid layer is therefore a key element in the design of a polymer-lipid drug carrier giving it unique properties.

The essence of the solution according to the invention for the polymer-lipid carrier is that the carrier is composed of a core containing polylactic-co-glycolic acid (PLGA) in an amount of 16.1 to 30.3% by weight and polyvinyl alcohol (PVA) in an amount of 60.6 to 75.5% by weight, and a core envelope containing a mixture of lipids: 1,2-dipalmitoyl-sn-glycero-3-phosphocholine (DDPC) in an amount ranging from 3.66% to 12.6% by weight, cholesterol in an amount ranging from 1.85 to 6.29% by weight and the ammonium salt 1,2-distearoyl-sn-glycero-3-phosphoethanolamine-N-[amino(polyethylene glycol)-2000 (DSPE-PEG(2000)NH2] in an amount ranging from 0.15 to 0.5% by weight.

Advantageously, PLGA is a 50:50 copolymer with an Mw of 24 000-38 000 g/mol.

Advantageously, PVA has a mass Mw of 30 000-70 000 g/mol.

The essence of the solution according to the invention in terms of the method is that:
in the first step, the core is obtained by preparing a clear aqueous solution of PVA at concentrations of 15 mg/ml to 20 mg/ml, followed by a solution of PLGA in dichloromethane at concentrations of 10 mg/ml to 20 mg/ml, at least one drug is then weighed out in the amount needed to make solutions with a concentration range of 0.43 mM to 4.30 mM and dissolved in the PLGA solution, after which both solutions are cooled to between 4° C. and 7° C. and then mixed in a volume ratio of 1:2, where two parts of aqueous PVA solution are equal to one part of PLGA solution in dichloromethane with the drug and cooled again to 4° C. to 7° C. and the whole is mixed to form an emulsion; the emulsion is poured into a crystalliser with a stirring element and the residue is washed with PBS buffer pH 7.4 or water at a volume ratio of the rinse solution (PBS or water) to the reaction mixture of 1:7.5 to 1:10 and also placed in the crystalliser, after which the dichloromethane is completely evaporated, the volume is then replenished with PBS 7.4 solution or water until a final PLGA concentration of 3.33 mg/ml to 6.67 mg/ml is obtained,
in a second step, the previously formed core is covered with DPPC/Cholesterol/DSPE-PEG(2000)NH2 liposomes (1.0/0.61/0.040), synthesised by the chosen method, and the lipid film formed is then hydrated with sterile saline NaCl 0.9% to a total lipid concentration of 10 mg/ml and dispersed by ultrasound to obtain a solution of DPPC/Cholesterol/DSPE-PEG(2000)NH2 liposomes as an intermediate for further synthesis; the liposomes obtained are then added to PVA/PLGA polymer cores not subjected to purification at a volume ratio of liposomes to polymer cores of 0.1:1 to 0.3:1, after which the whole is heated to 45 to 60° C. for 2 h to 4 h with stirring at 200 to 600 RPM, and the resulting PLGA/PVA/LIPO nanoparticles are left at 4° C. for 12 to 24 h and purified by washing twice with PBS buffer 7.4.

Advantageously, PLGA is a 50:50 copolymer with an Mw of 24 000-38 000 g/mol.

Advantageously, PVA has a mass Mw of 30 000-70 000 g/mol.

Advantageously, 0.1 M PBS buffer pH 7.4 is added to the PVA solution.

Advantageously, the inner core polymer is a copolymer of poly(L-lactide-co-glycolide)—PLGA.

Advantageously, the outer polymer of the core is poly(vinyl alcohol) PVA.

Favourably, the drug introduced into the core is Staurosporine between 5 and 11.5 mg.

Favourably, the drug introduced into the core is 17AAG (Tanespimycin) between 5 and 11.5 mg.

Favourably, Staurosporine and 17AAG (Tanespimycin) are introduced into the core with a concentration ratio of 17AAG (Tanespimycin) to Staurosporine between 0.62 and 9.5.

Advantageously, the evaporation of dichloromethane is carried out using a magnetic stirrer at a speed of 200 to 600 RPM at a temperature of 37° C. to 40° C.

Advantageously, the emulsion is obtained by agitation with an ultrasonic probe from 1 minute to 2 minutes at an amplitude of 70%-90%.

Advantageously, in order to control the quality of the manufactured PLGA/PVA polymer core, a measurement of the size of the nanoparticles is performed using a dynamic light scattering method.

Advantageously, DPPC/Cholestrol/DSPE-PEG(2000)NH2 liposomes are synthesised by a hydration method, preparing a solution of the lipid mixture with a concentration of: DPPC equal to 96.0 mM, Cholesterol equal to 58.8 mM, DSPE-PEG(2000)NH2 equal to 3.84 mM in chloroform.

Advantageously, DPPC/Cholestrol/DSPE-PEG(2000)NH2 liposomes are synthesised by an ethanol injection method, preparing a solution of the lipid mixture with a concentration of: DPPC equal to 96.0 mM, Cholesterol equal to 58.8 mM, DSPE-PEG(2000)NH2 equal to 3.84 mM in ethanol.

Advantageously, in the hydration method, the solvent is evaporated using a vacuum evaporator or vacuum dryer.

Advantageously, in the ethanol injection method, a solution of lipids in ethanol is dropped into sterile saline mixed with a magnetic stirrer to obtain a solution of DPPC/Cholestrol/DSPE-PEG(2000)NH2 liposomes with a total lipid concentration of 10 mg/ml as an intermediate for further synthesis.

Advantageously, the purification of nanoparticles is carried out in a high-speed centrifuge at 15,000 RPM for 2 to 6 min.

Advantageously, after each addition of PBS 7.4, the nanoparticles are dispersed using ultrasound for not less than 5 seconds.

Advantageously, in order to stabilise the nanoparticles, a 40% solution of non-ionic surfactant in a volume ratio of 0.02:1 (surfactant:nanoparticles) or glucose is added after the last centrifugation until a concentration of 2 mg/ml is reached, after which the solution is stirred.

Advantageously, the drug content, i.e. the % EE, is determined by UV-Vis spectrophotometry for single or a mixture of drugs by dissolving PLGA/PVA/LIPO nanoparticles in DMSO with HCL according to the scheme: 25-75 uL sample, 50 uL 0.3M HCl, 25-75 uL PBS 7.4 and 850 uL DMSO, or by LC-MS method.

The main advantage of the solutions according to the inventions is that the combination of polymers with lipids used in the nanoparticles allows: loading of single drugs such as 17AAG (Tanespimycin) and Staurosporine, as well as their combinations, at a level of 32% to 80%; sustained release of the drug or drug combination, prevention of excessive drug leakage and aggregation of the carrier in the presence of plasma proteins. In addition, it allows a range of 17AAG to Staurosporine concentration ratios from 0.62 to 9.5. The lipid bilayer coating with polyethyleneglycol used allows the prevention of nanoparticle aggregation in the presence of proteins and ensures the stability of the nanoparticles at 37° C. over 96 h. The size of the PLGA/PVA/LIPO nanoparticles ranging from 209 nm to 388 nm ensures selective, passive transport of the drug into tumour tissues due to the effect of increased permeability and EPR retention. In addition, the additional modification of polyethylene glycol with amine groups giving the nanoparticles a low positive potential of 6.5 mV to 9.5 mV allows for enhanced anti-tumour efficacy, better accumulation and penetration of the carrier in tumour tissues and better cellular uptake compared to inert and negatively charged nanoparticles (https://doi.org/10.1016/j.nantod.2016.04.008). With such a developed structure, the developed nanoparticles have a small diameter and low PDI polydispersity index.

In summary, the developed nanoparticles provide a high loading rate, reduced initial drug release and sustained drug release, a positive zeta potential for carrier accumulation in tumour tissue and long-term stability of the nanoparticle in the presence of plasma proteins over 96 h.

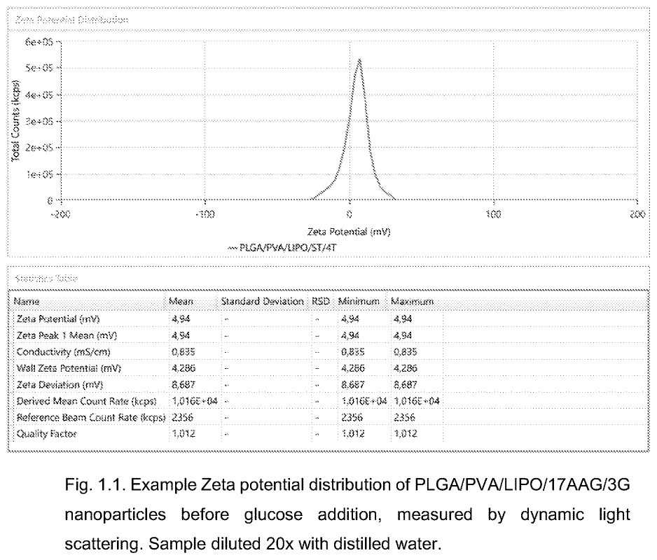

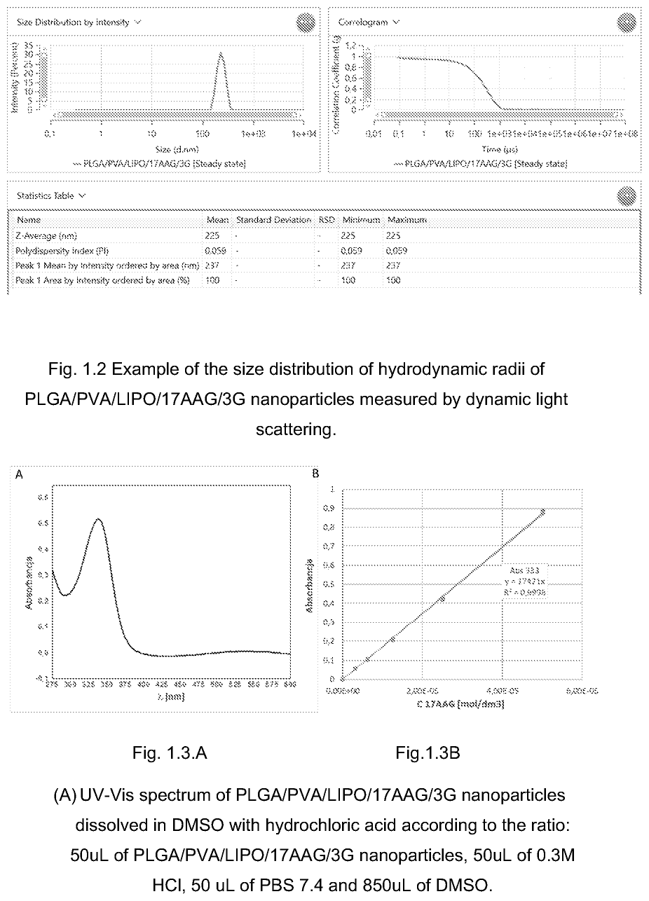

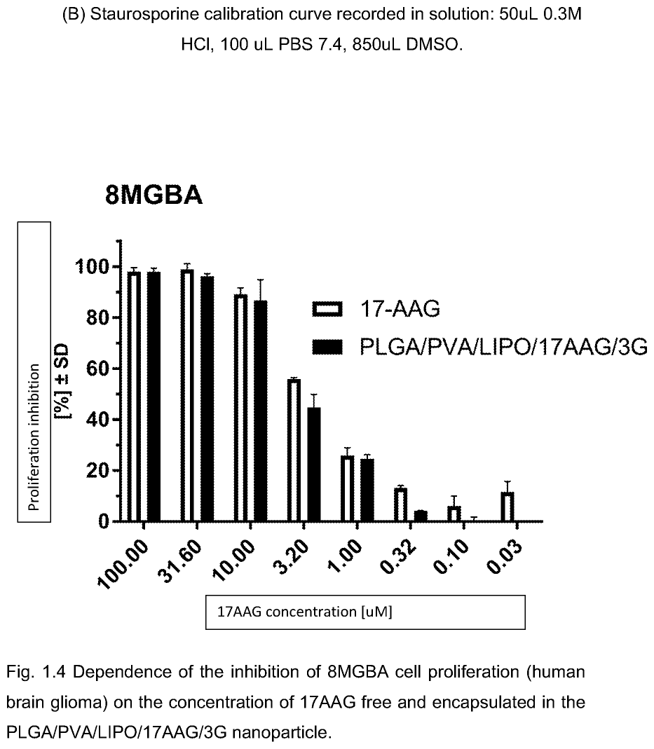

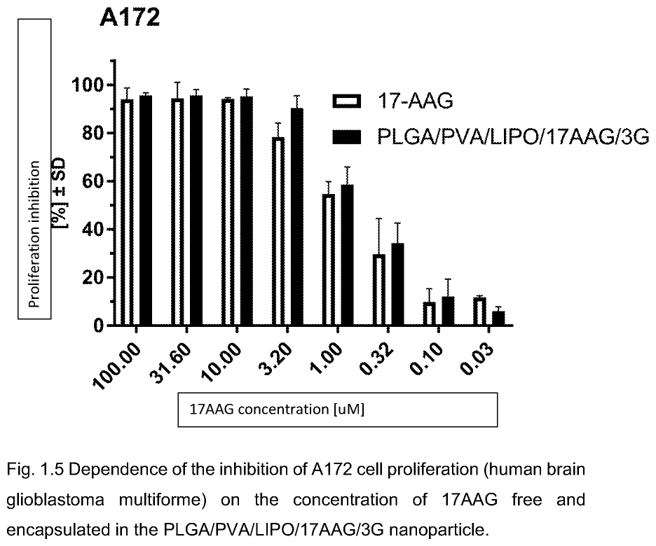

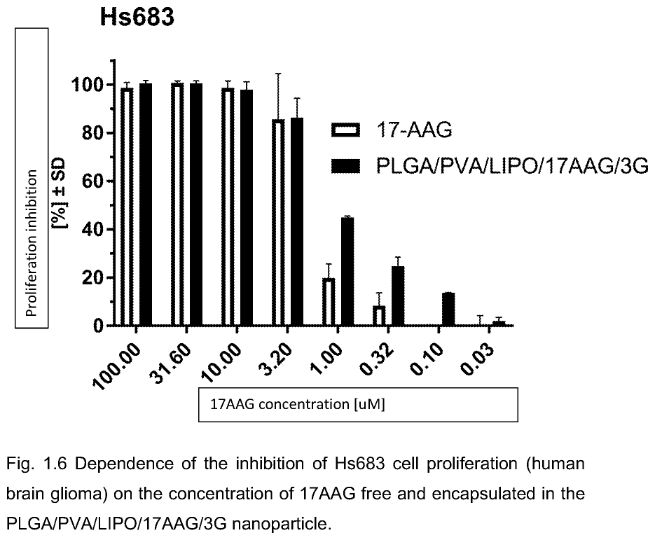

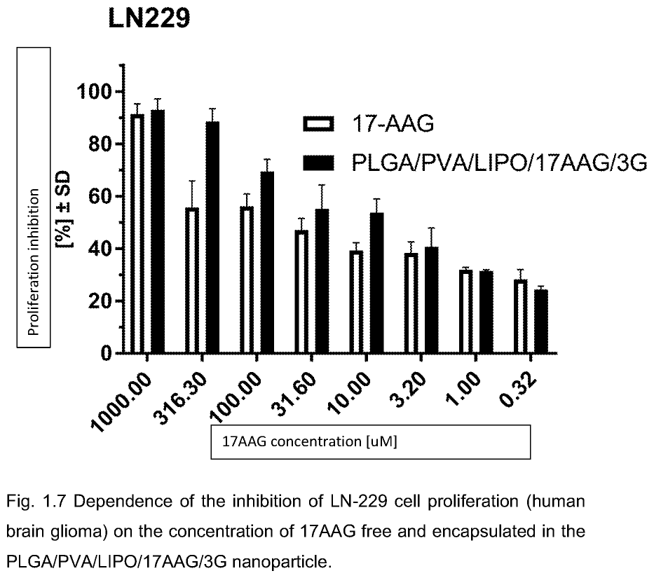

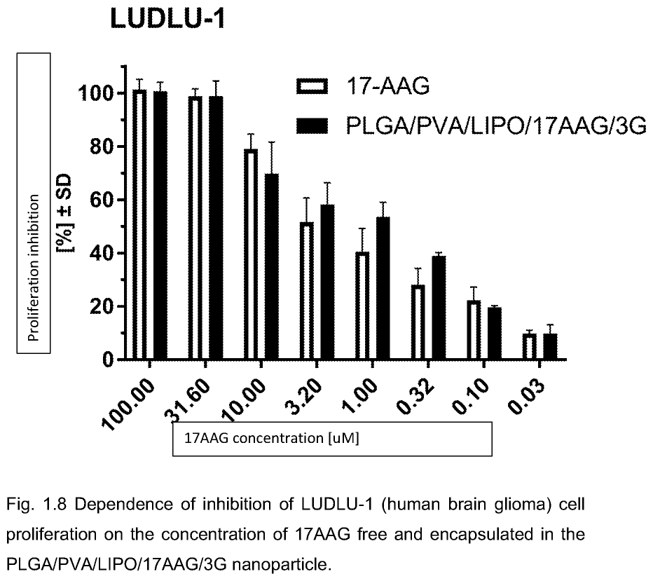

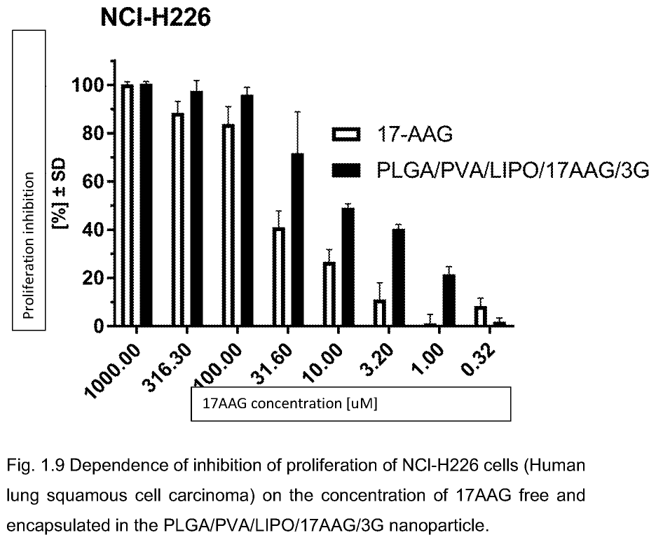

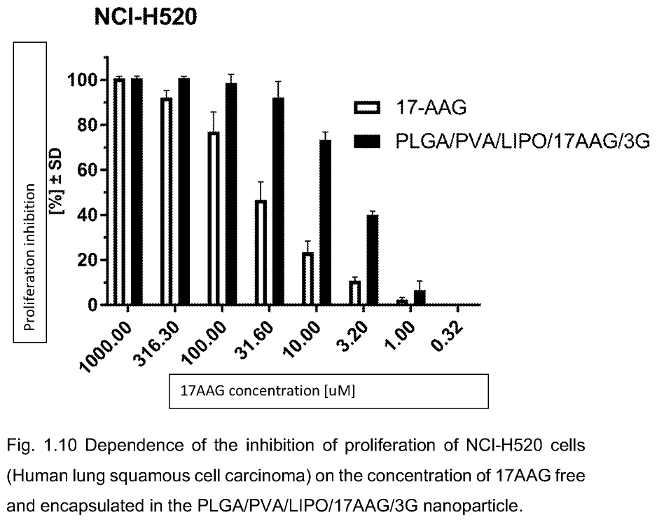

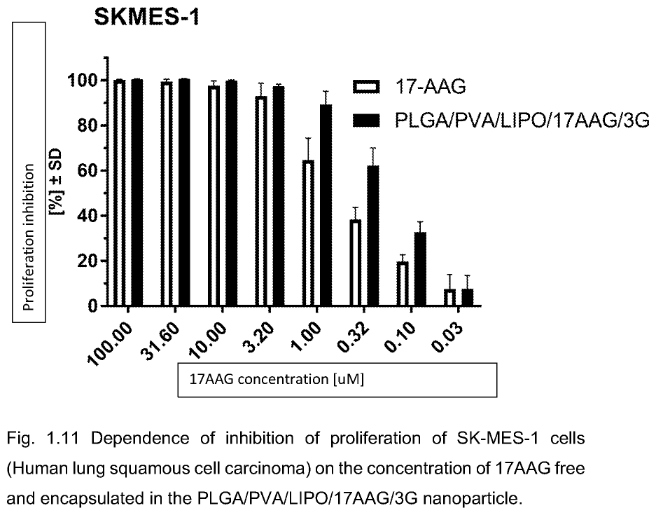

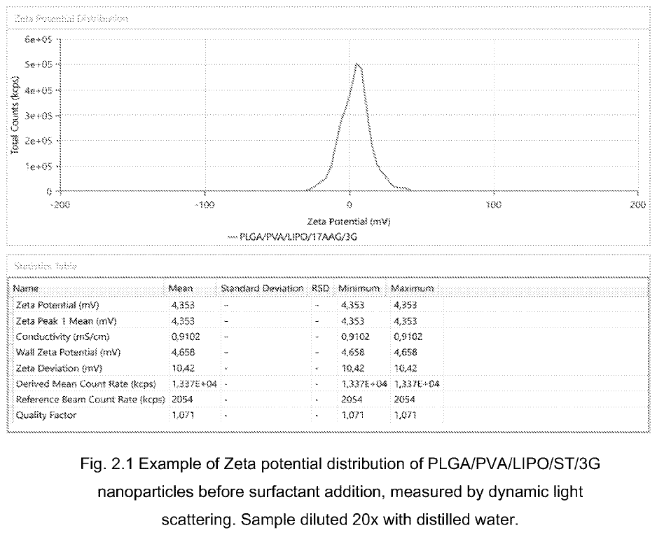

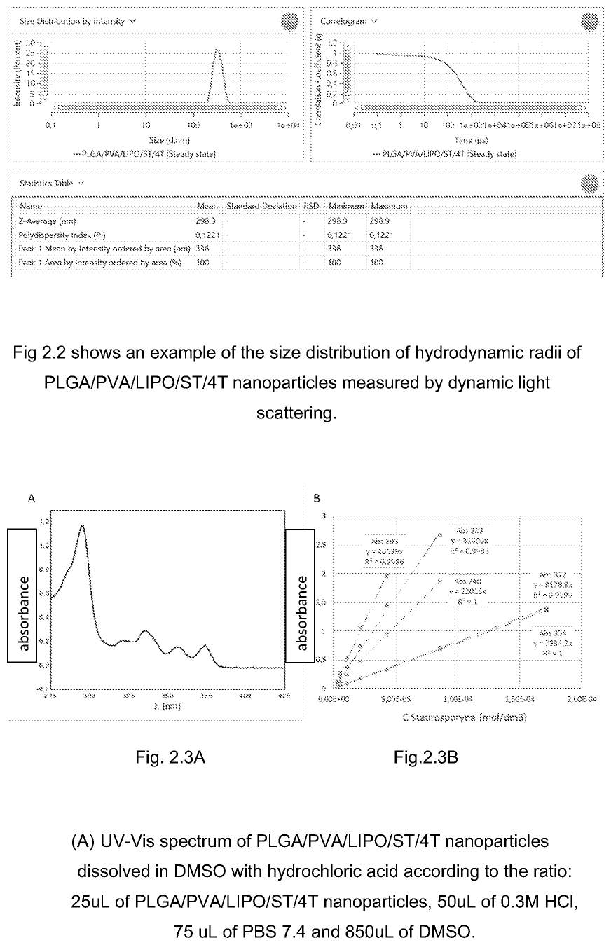

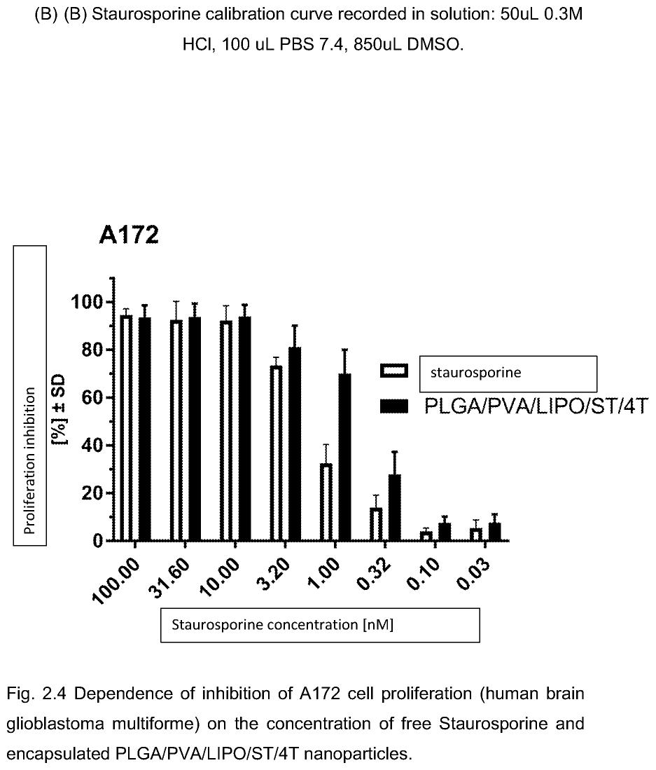

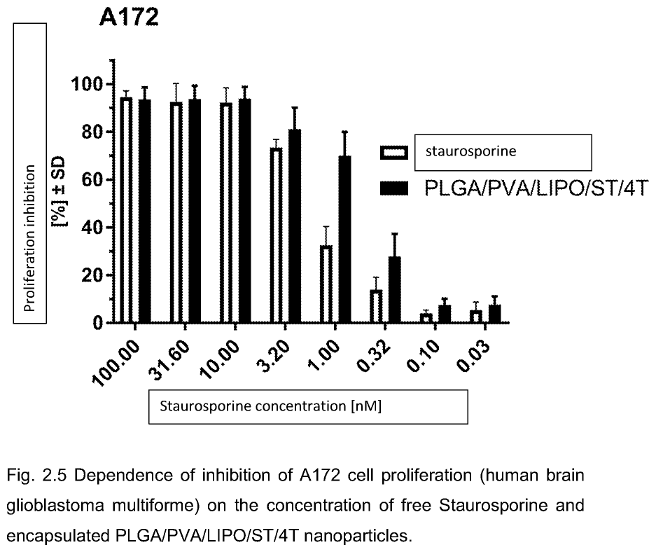

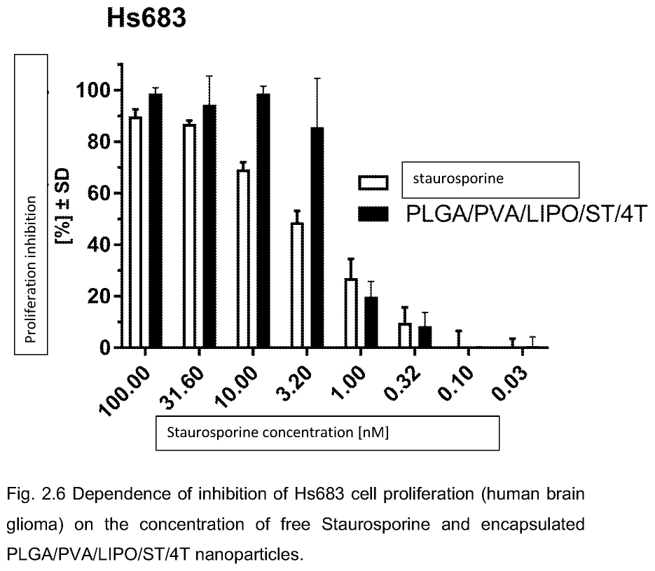

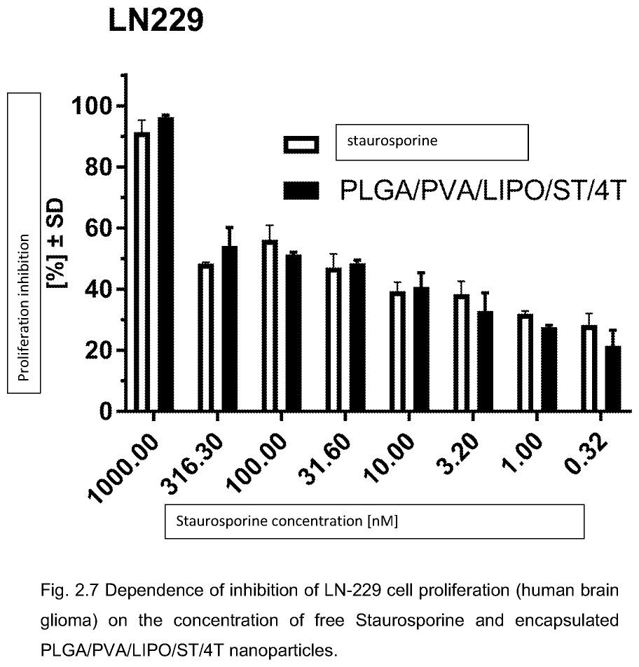

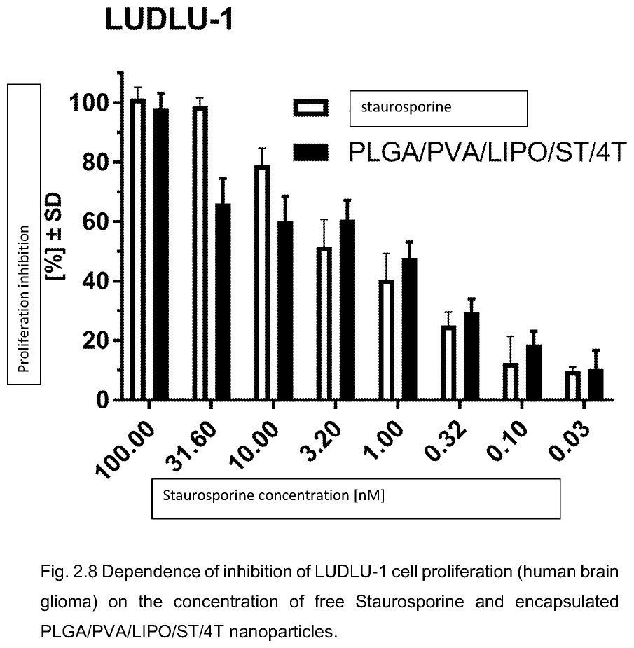

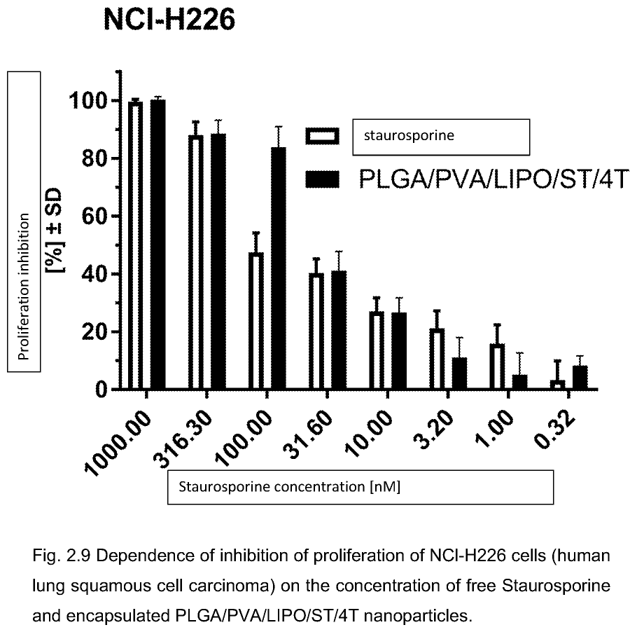

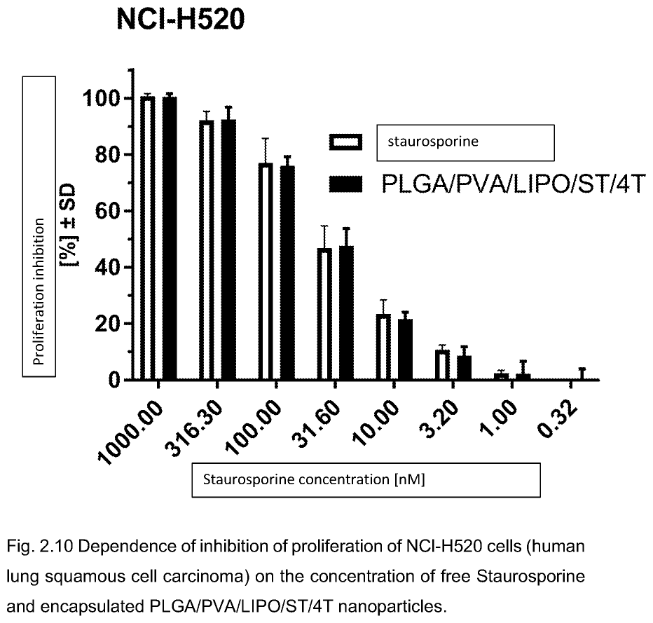

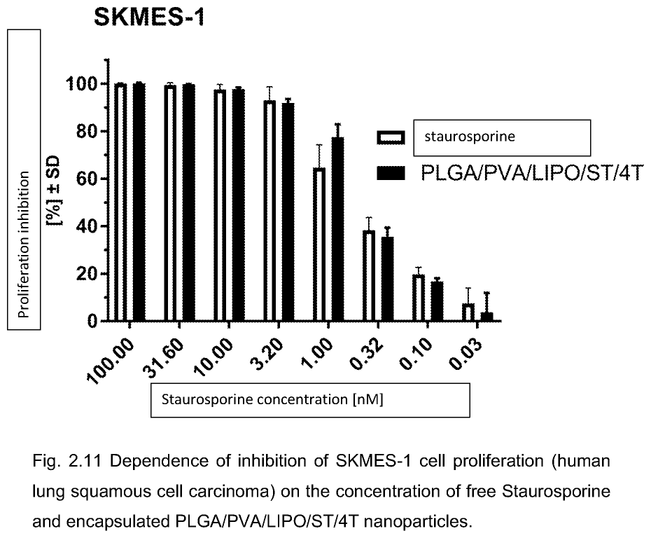

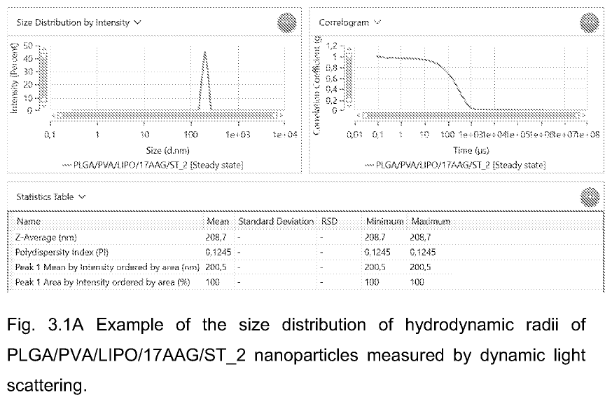

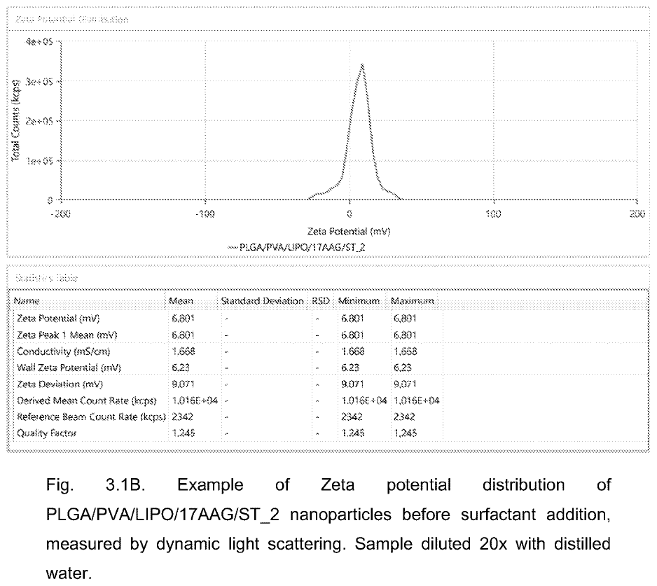

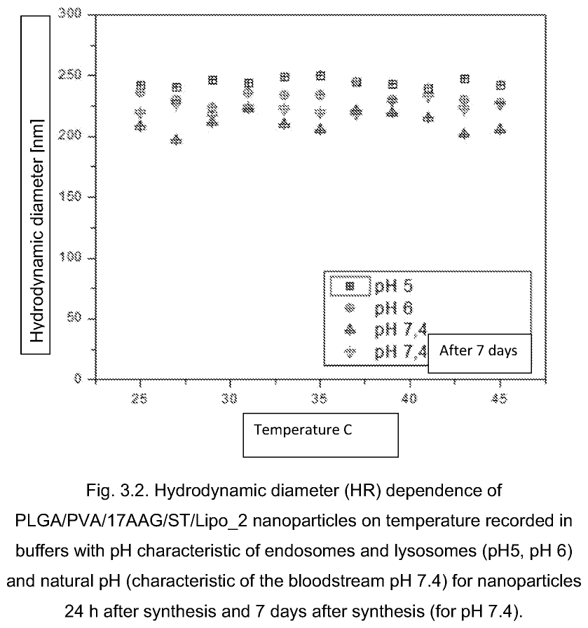

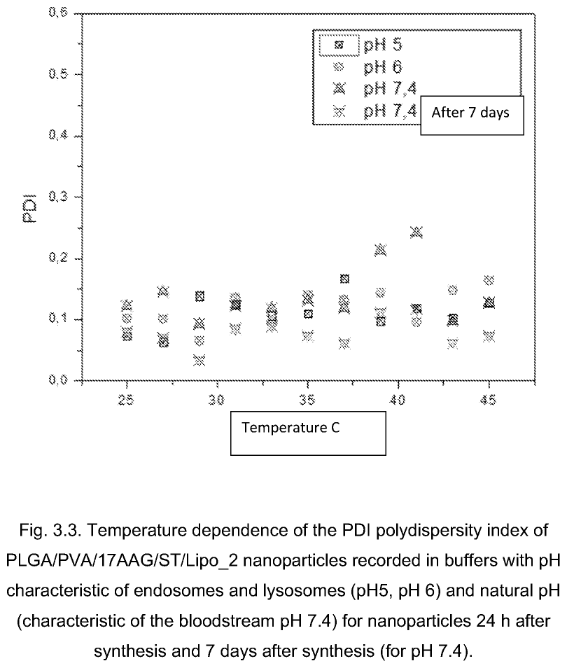

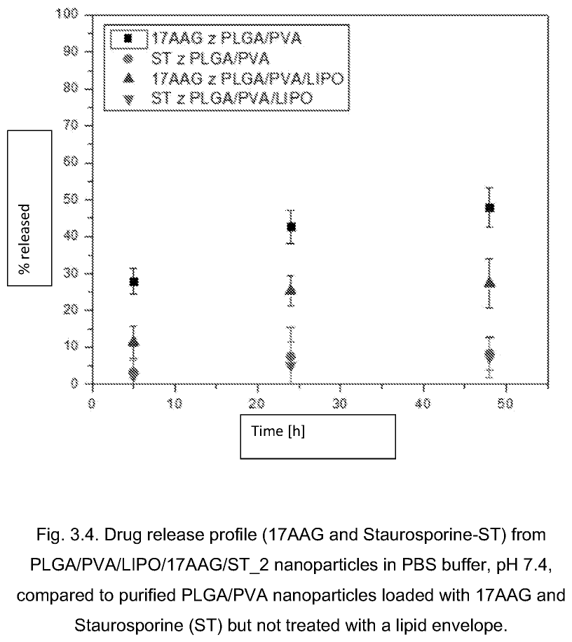

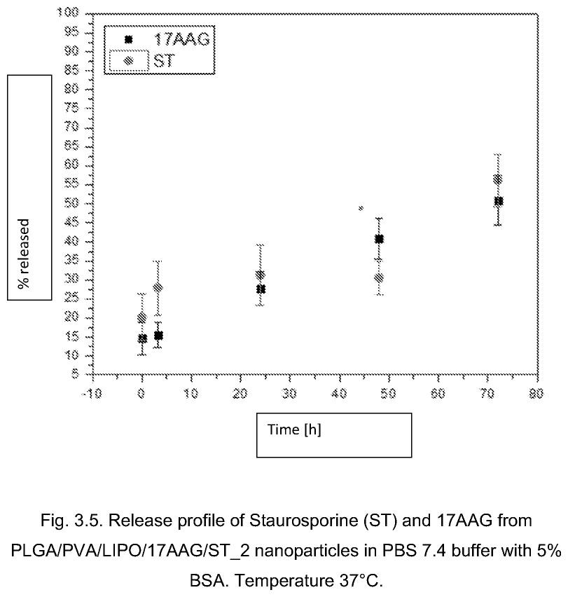

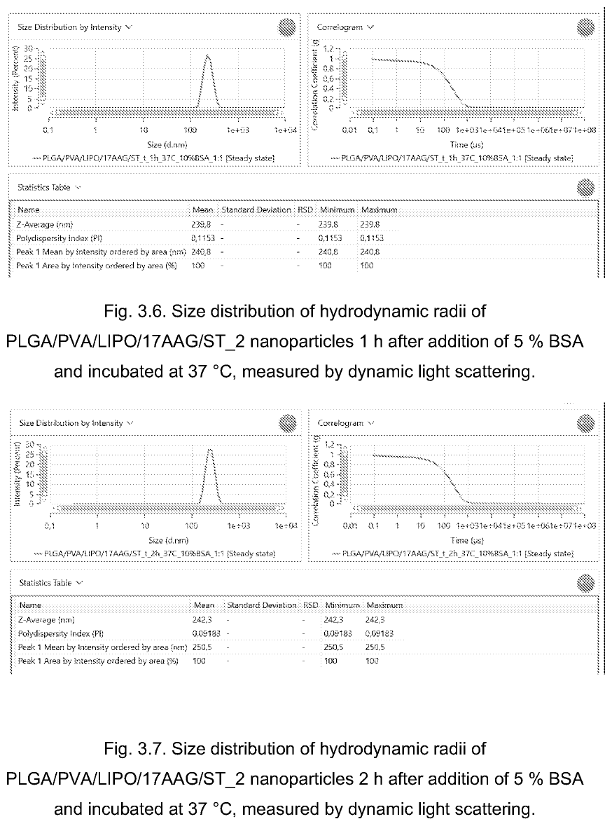

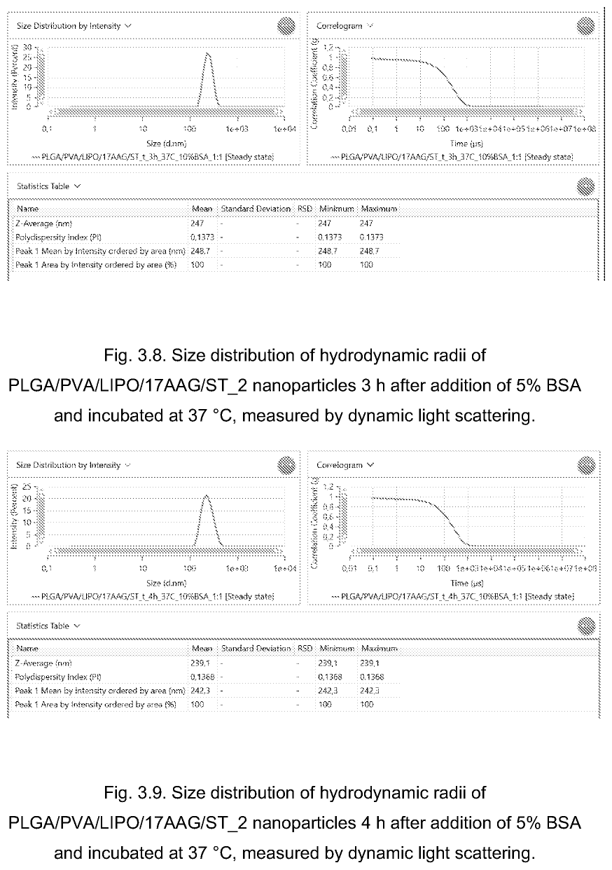

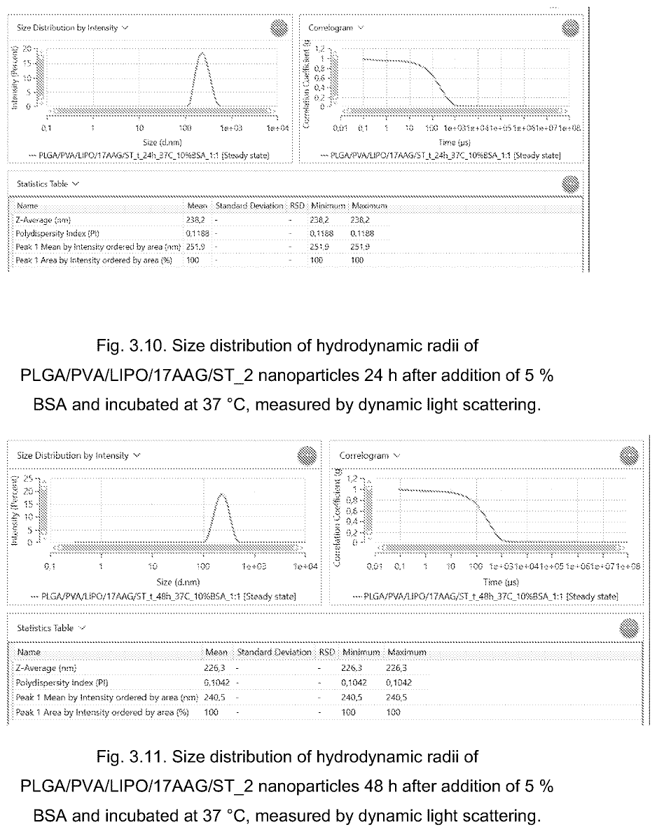

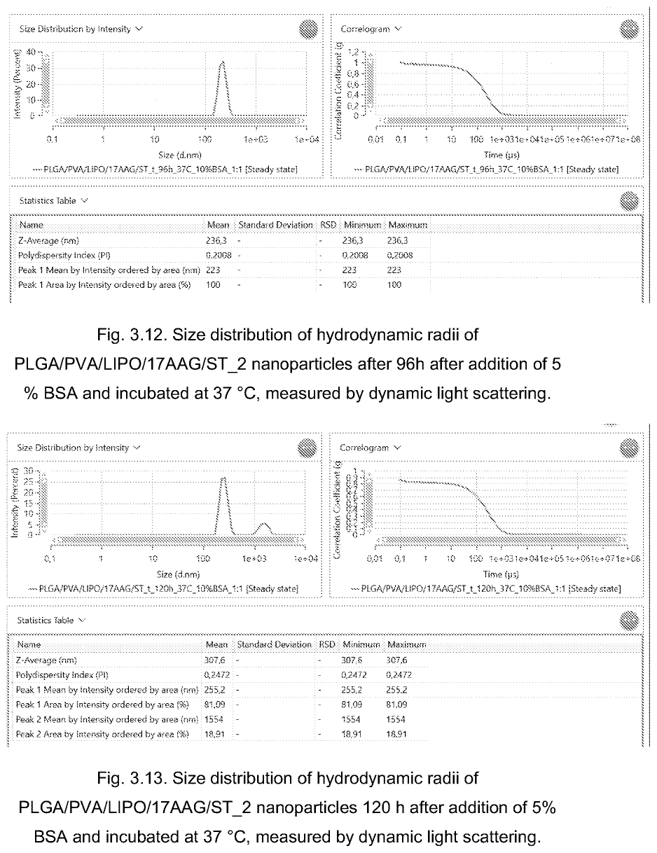

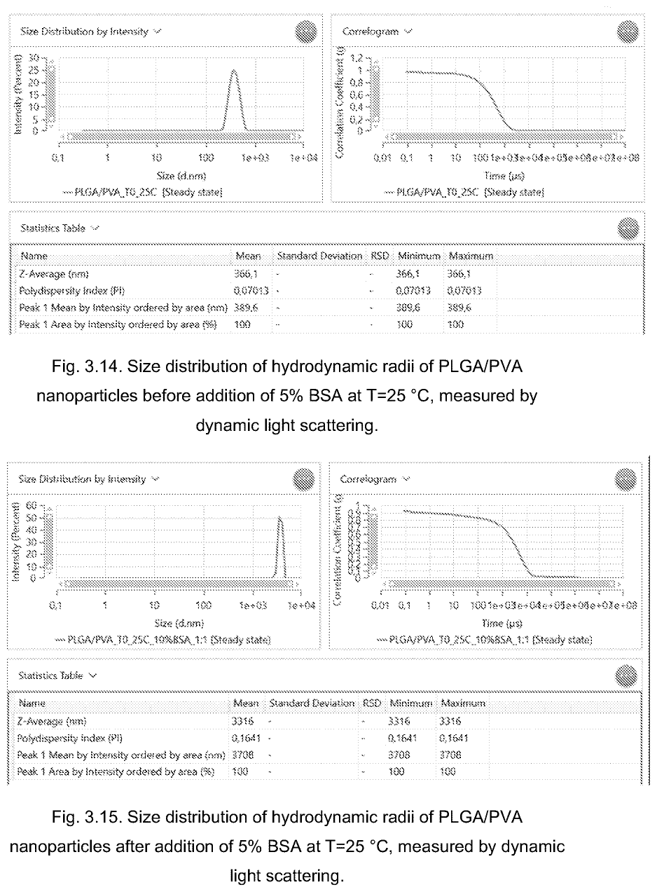

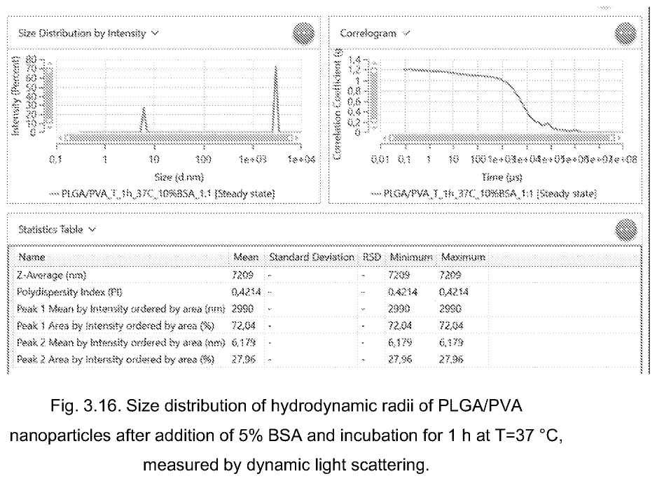

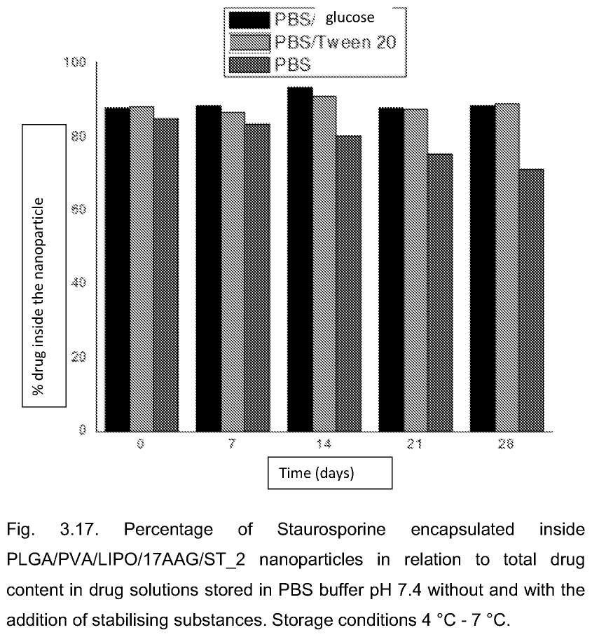

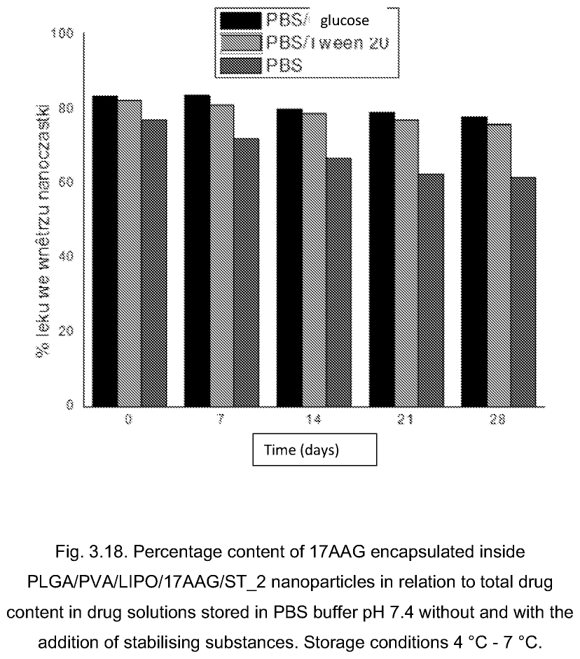

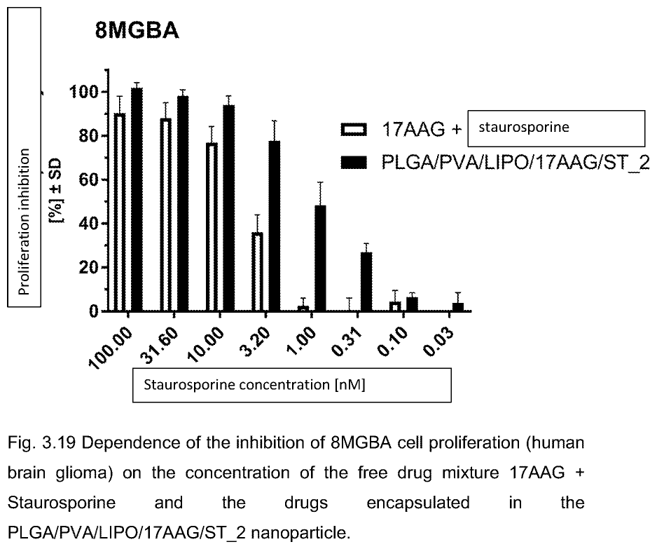

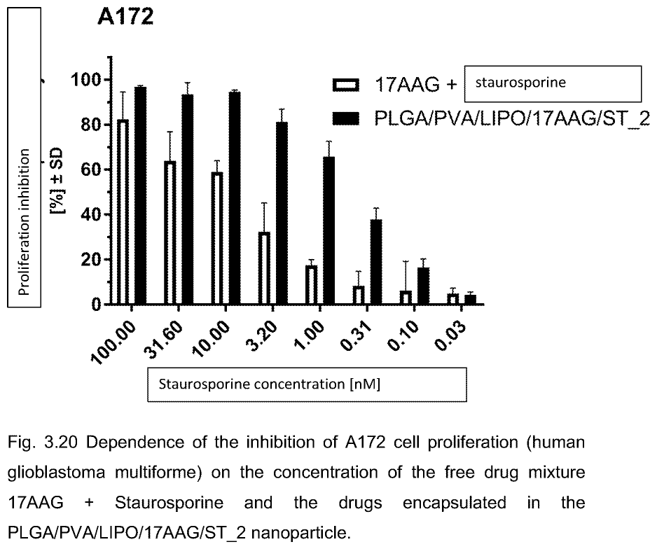

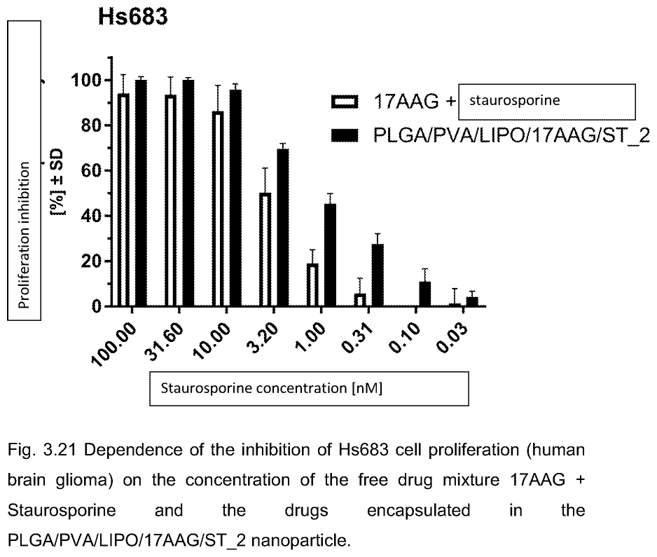

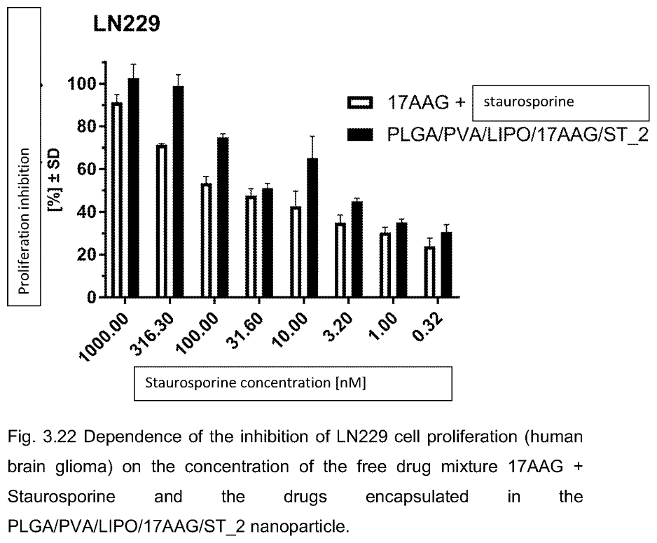

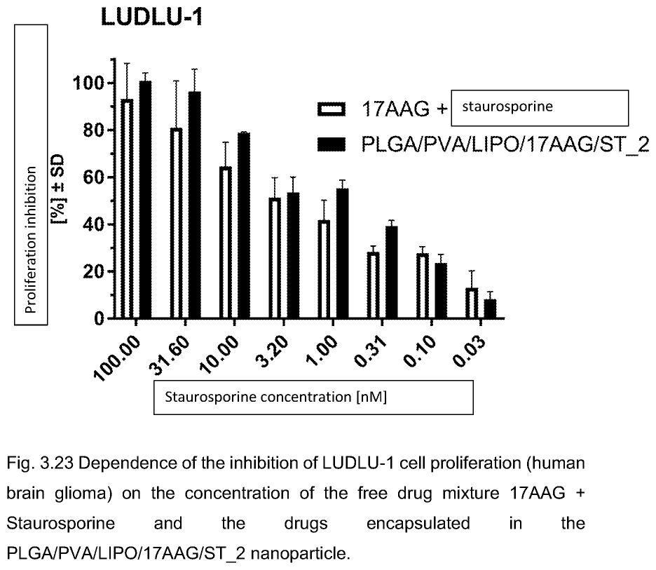

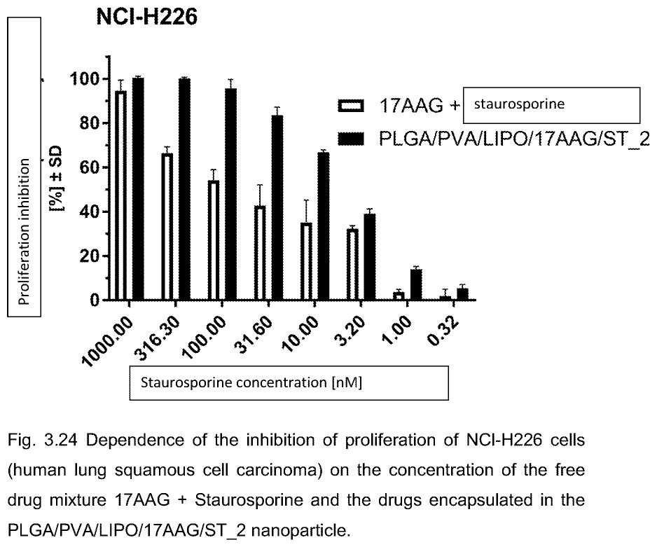

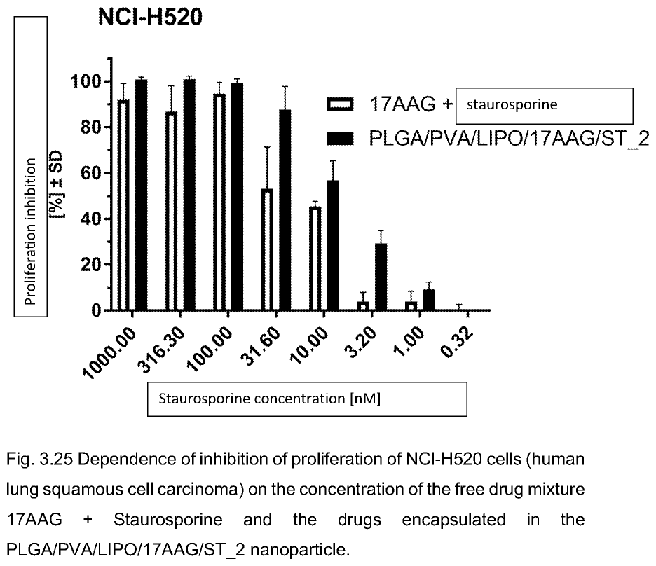

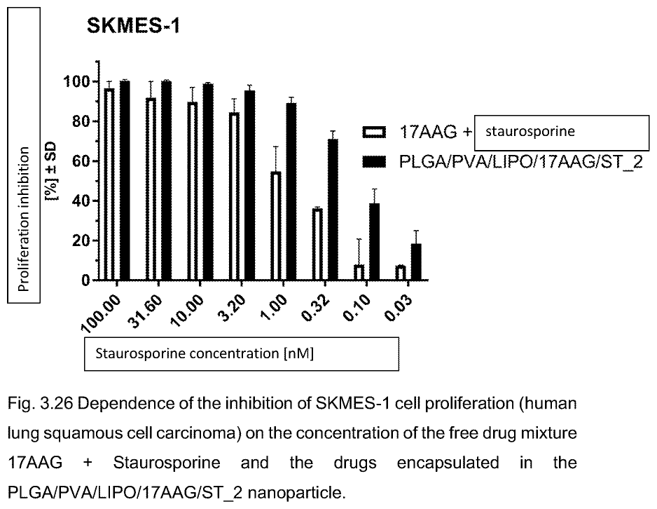

The solutions according to the inventions are illustrated by the following manufacturing examples and drawings, where FIG. 1.1 shows an example of the size distribution of hydrodynamic rays of PLGA/PVA/LIPO/17AAG/1G nanoparticles measured by dynamic light scattering, FIG. 1.2—an example of the size distribution of hydrodynamic rays of PLGA/PVA/LIPO/17AAG/3G nanoparticles measured by dynamic light scattering, FIG. 1.3.A—UV-Vis spectrum of PLGA/PVA/LIPO/17AAG/3G nanoparticles dissolved in DMSO with hydrochloric acid according to the ratio: 50 uL of PLGA/PVA/LIPO/17AAG/3G nanoparticles, 50 uL of 0.3M HCl, 50 uL of PBS 7.4 and 850 uL of DMSO, FIG. 1.3.B—Staurosporine calibration curve recorded in solution: 50 uL 0.3M HCl, 100 uL PBS 7.4, 850 uL DMSO, FIG. 1.4—Dependence of inhibition of 8MGBA (human brain glioma) cell proliferation on the concentration of 17AAG free and encapsulated in the PLGA/PVA/LIPO/17AAG/3G nanoparticle, FIG. 1.5—Dependence of the inhibition of A172 cell proliferation (human brain glioma multiforme) on the concentration of 17AAG free and encapsulated in the PLGA/PVA/LIPO/17AAG/3G nanoparticle, FIG. 1.6—Dependence of proliferation inhibition of Hs683 cells (human brain glioma) on the concentration of 17AAG free and encapsulated in PLGA/PVA/LIPO/17AAG/3G nanoparticle, FIG. 1.7—Dependence of proliferation inhibition of LN-229 cells (human brain glioma) on the concentration of 17AAG free and encapsulated in PLGA/PVA/LIPO/17AAG/3G nanoparticle, FIG. 1.8—Dependence of inhibition of LUDLU-1 cell proliferation (human brain glioma) on the concentration of 17AAG free and encapsulated in PLGA/PVA/LIPO/17AAG/3G nanoparticle, FIG. 1.9—Dependence of inhibition of NCI-H226 cell proliferation (human lung squamous cell carcinoma) on the concentration of 17AAG free and encapsulated in PLGA/PVA/LIPO/17AAG/3G nanoparticle, FIG. 1.10—Dependence of inhibition of NCI-H520 cell proliferation (Human lung squamous cell carcinoma) on the concentration of 17AAG free and encapsulated in the PLGA/PVA/LIPO/17AAG/3G nanoparticle, FIG. 1.11—Dependence of inhibition of SK-MES-1 cell proliferation (Human lung squamous cell carcinoma) on the concentration of 17AAG free and encapsulated in the PLGA/PVA/LIPO/17AAG/3G nanoparticle, FIG. 2.1 Example of Zeta potential distribution of PLGA/PVA/LIPO/ST/3G nanoparticles before surfactant addition, measured by dynamic light scattering. Sample diluted 20× with distilled water, FIG. 2.1—example size distribution of hydrodynamic rays of PLGA/PVA/LIPO/ST/4T nanoparticles measured by dynamic light scattering, FIG. 2.2—shows example size distribution of hydrodynamic rays of PLGA/PVA/LIPO/ST/4T nanoparticles measured by dynamic light scattering, FIG. 2.3A—UV-Vis spectrum of PLGA/PVA/LIPO/ST/4T nanoparticles dissolved in DMSO with hydrochloric acid according to the ratio: 25 uL of PLGA/PVA/LIPO/ST/4T nanoparticles, 50 uL of 0.3M HCl, 75 uL of PBS 7.4 and 850 uL of DMSO, FIG. 2.3B—Staurosporine calibration curve recorded in solution: 50 uL 0.3M HCl, 100 uL PBS 7.4, 850 uL DMSO, FIG. 2.4—Dependence of inhibition of A172 cell proliferation (human brain glioblastoma multiforme) on the concentration of free Staurosporine and encapsulated PLGA/PVA/LIPO/ST/4T nanoparticles, FIG. 2.5—Dependence of inhibition of A172 cell proliferation (human brain glioblastoma multiforme) on the concentration of free Staurosporine and encapsulated in PLGA/PVA/LIPO/ST/4T nanoparticle. FIG. 2.6—Dependence of proliferation inhibition of Hs683 cells (human brain glioma) on the concentration of free Staurosporine and encapsulated in PLGA/PVA/LIPO/ST/4T nanoparticle, FIG. 2.7—Dependence of proliferation inhibition of LN-229 cells (human brain glioma) on the concentration of free Staurosporine and encapsulated in PLGA/PVA/LIPO/ST/4T nanoparticle, FIG. 2.8—Dependence of proliferation inhibition of LUDLU-1 cells (human brain glioma) on the concentration of free Staurosporine and encapsulated in PLGA/PVA/LIPO/ST/4T nanoparticle, FIG. 2.9—Dependence of proliferation inhibition of NCI-H226 cells (human lung squamous cell carcinoma) on the concentration of free Staurosporine and encapsulated in PLGA/PVA/LIPO/ST/4T nanoparticle, FIG. 2.10—Dependence of inhibition of NCI-H520 cell proliferation (human lung squamous cell carcinoma) on the concentration of free Staurosporine and encapsulated in PLGA/PVA/LIPO/ST/4T nanoparticle, FIG. 2.11—Dependence of SKMES-1 cell proliferation inhibition (human lung squamous cell carcinoma) on the concentration of free Staurosporine and encapsulated in PLGA/PVA/LIPO/ST/4T nanoparticle, FIG. 3.1A—Example size distribution of hydrodynamic radii of PLGA/PVA/LIPO/17AAG/ST_2 nanoparticles measured by dynamic light scattering, FIG. 3.1B—Example Zeta potential distribution of PLGA/PVA/LIPO/17AAG/ST_2 nanoparticles before surfactant addition, measured by dynamic light scattering. FIG. 3.2—plot of hydrodynamic diameter (HR) of PLGA/PVA/17AAG/ST/Lipo_2 nanoparticles against temperature, recorded in buffers with pH characteristic of endosomes and lysosomes (pH 5, pH 6) and natural pH (characteristic of bloodstream pH 7.4) for nanoparticles 24 h after synthesis and 7 days after synthesis (for pH 7.4), FIG. 3.3. 3.3.—plot of the temperature dependence of the PDI polydispersity index of PLGA/PVA/17AAG/ST/Lipo_2 nanoparticles recorded in buffers of pH characteristic of endosomes and lysosomes (pH 5, pH 6) and natural pH (characteristic of the bloodstream pH 7.4) for nanoparticles 24 h after synthesis and 7 days after synthesis (for pH 7.4), FIG. 3.4.—drug release profile (17AAG and Staurosporine-ST) from PLGA/PVA/LIPO/17AAG/ST_2 nanoparticles in PBS buffer pH 7.4 compared to purified PLGA/PVA nanoparticles loaded with 17AAG and Staurosporine (ST) but not subjected to lipid coating of the nanoparticles, FIG. 3.5.—Release profile of Staurosporine (ST) and 17AAG from PLGA/PVA/LIPO/17AAG/ST_2 nanoparticles in PBS 7.4 buffer with 5% BSA at 37° C., FIG. 3.6.—size distribution of hydrodynamic radii of PLGA/PVA/LIPO/17AAG/ST_2 nanoparticles 1 h after addition of 5% BSA and incubated at 37° C., measured by dynamic light scattering, FIG. 3.7.—Size distribution of hydrodynamic radii of PLGA/PVA/LIPO/17AAG/ST_2 nanoparticles 2 h after addition of 5% BSA and incubated at 37° C., measured by dynamic light scattering, FIG. 3.8 size distribution of hydrodynamic radii of PLGA/PVA/LIPO/17AAG/ST_2 nanoparticles 3 h after addition of 5% BSA and incubated at 37° C., measured by dynamic light scattering, FIG. 3.9.—size distribution of hydrodynamic radii of PLGA/PVA/LIPO/17AAG/ST_2 nanoparticles after 4 h of 5% BSA addition and incubated at 37° C., measured by dynamic light scattering, FIG. 3.10.—size distribution of hydrodynamic radii of PLGA/PVA/LIPO/17AAG/ST_2 nanoparticles after 24 h of 5% BSA addition and incubated at 37° C., measured by dynamic light scattering, FIG. 3.11.—size distribution of hydrodynamic radii of PLGA/PVA/LIPO/17AAG/ST_2 nanoparticles after 48 h after addition of 5% BSA and incubated at 37° C., measured by dynamic light scattering, FIG. 3.12.—Size distribution of hydrodynamic rays of PLGA/PVA/LIPO/17AAG/ST_2 nanoparticles 96 h after addition of 5% BSA and incubated at 37° C., measured by dynamic light scattering, FIG. 3.13.—Size distribution of hydrodynamic rays of PLGA/PVA/LIPO/17AAG/ST_2 nanoparticles 120 h after addition of 5% BSA and incubated at 37° C., measured by dynamic light scattering, FIG. 3.14.—size distribution of hydrodynamic radii of PLGA/PVA nanoparticles before addition of 5% BSA at T=25° C., measured by dynamic light scattering, FIG. 3.15.—size distribution of hydrodynamic radii of PLGA/PVA nanoparticles after addition of 5% BSA at T=25° C., measured by dynamic light scattering, FIG. 3.16.—Distribution of the size of hydrodynamic radii of PLGA/PVA nanoparticles after addition of 5% BSA and incubation for 1 hour at T=37° C., measured by dynamic light scattering, FIG. 3.17.—plot of the percentage of Staurosporine encapsulated inside PLGA/PVA/LIPO/17AAG/ST_2 nanoparticles against total drug content in drug solutions stored in PBS buffer pH 7.4 without and with addition of stabilising substances, FIG. 3.18—Graph of the percentage of 17AAG encapsulated inside PLGA/PVA/LIPO/17AAG/ST_2 nanoparticles in relation to the total drug content in drug solutions stored in PBS buffer at pH 7.4 without and with the addition of stabilising substances, FIG. 3.19—Dependence of the inhibition of 8MGBA cell proliferation (human brain glioma) on the concentration of the free drug mixture 17AAG+Staurosporine and drugs encapsulated in the PLGA/PVA/LIPO/17AAG/ST_2 nanoparticle, FIG. 3.20—Dependence of the inhibition of A172 cell proliferation (human brain glioblastoma multiforme) on the concentration of the mixture of free drugs 17AAG+Staurosporine and drugs encapsulated in the PLGA/PVA/LIPO/17AAG/ST_2 nanoparticle, FIG. 3.21—Dependence of the inhibition of Hs683 cell proliferation (human brain glioma) on the concentration of the mixture of free drugs 17AAG+Staurosporine and drugs encapsulated in the PLGA/PVA/LIPO/17AAG/ST_2 nanoparticle, FIG. 3.22—Dependence of inhibition of LN229 cell proliferation (human brain glioma) on the concentration of the mixture of free drugs 17AAG+Staurosporine and drugs encapsulated in the PLGA/PVA/LIPO/17AAG/ST_2 nanoparticle, FIG. 3.23 Dependence of the inhibition of LUDLU-1 (human brain glioma) cell proliferation on the concentration of the mixture of free drugs 17AAG+Staurosporine and drugs encapsulated in the PLGA/PVA/LIPO/17AAG/ST_2 nanoparticle, FIG. 3.24—Concentration dependence of inhibition of proliferation of NCI-H226 cells (human lung squamous cell carcinoma) on the concentration of the mixture of free drugs 17AAG+Staurosporine and drugs encapsulated in the PLGA/PVA/LIPO/17AAG/ST_2 nanoparticle, FIG. 3.25—Concentration dependence of proliferation inhibition of NCI-H520 cells (human lung squamous cell carcinoma) on the concentration of the mixture of free drugs 17AAG+Staurosporine and drugs encapsulated in the PLGA/PVA/LIPO/17AAG/ST_2 nanoparticle, FIG. 3.26—Concentration dependence of the inhibition of SKMES-1 cell proliferation (human lung squamous cell carcinoma) on the concentration of the mixture of free drugs 17AAG+Staurosporine and drugs encapsulated in the PLGA/PVA/LIPO/17AAG/ST_2 nanoparticle.

TABLE 1
Mass composition and % ww of PLGA/PVA/LIPO carriers

| | PLGA/PVA/LIPO CARRIER | | | | | | | | | |
|---|---|---|---|---|---|---|---|---|---|---|
| | PLGA | | PVA | | DPPC | | Cholesterol | | DSPE-PEG(2000)NH2 | |
| | | % | | % | | % | | % | | % |
| L.p | [mg]. | w/w | [mg]. | w/w | [mg]. | w/w | [mg]. | w/w | [mg]. | w/w |
| 1 | 50 | 21.7 | 150 | 65.2 | 19.5 | 8.48 | 9.75 | 4.24 | 0.78 | 0.34 |
| 2 | 100 | 30.3 | 200 | 60.6 | 19.5 | 5.91 | 9.75 | 2.95 | 0.78 | 0.24 |
| 3 | 50 | 18.9 | 200 | 75.5 | 9.7 | 3.66 | 4.9 | 1.85 | 0.39 | 0.15 |
| 4 | 50 | 16.1 | 200 | 64.5 | 39 | 12.5 | 19.5 | 6.29 | 1.56 | 0.50 |
| 5 | 80 | 25.8 | 200 | 64.5 | 19.5 | 6.29 | 9.75 | 3.14 | 0.78 | 0.25 |

TABLE 2
Bulk composition of PLGA/PVA/LIPO nanoparticles together with the solvents
used to form the emulsion and the addition of drugs and stabiliser.

| | | | | | | | | | Stabiliser - |
|---|---|---|---|---|---|---|---|---|---|
| | | | | | | | | Organic | Non-ionic |
| | | | | | DSPE- | | | phase/ | surfactant/ |
| | PLGA | PVA | DPPC | Cholesterol | PEG(2000)NH2 | 17AAG | ST | water | Glucose/ |
| L.p. | [mg]. | [mg]. | [mg]. | [mg]. | [mg]. | [mg]. | [mg] | phase | None (—) |
| PLGA/PVA/ | 50.0 | 150.0 | 19.5 | 9.75 | 0.78 | 10 | 8.1 | CH₂ | (—) |
| LIPO/17AAG/ | | | | | | | | Cl₂/ | |
| ST_1 | | | | | | | | HO₂ | |
| PLGA/PVA/ | 100.0 | 200.0 | 19.5 | 9.75 | 0.78 | 11.5 | 9.5 | CH₂ | (—) |
| LIPO/17AAG/ | | | | | | | | Cl₂/ | |
| ST_2 | | | | | | | | PBS | |
| | | | | | | | | 7.4 | |
| PLGA/PVA/ | 50.0 | 200.0 | 9.7 | 4.9 | 0.39 | 10 | 8.1 | CH₂ | (—) |
| LIPO/17AAG/ | | | | | | | | Cl₂/ | |
| ST_3 | | | | | | | | HO₂ | |
| PLGA/PVA/ | 50.0 | 200.0 | 39.0 | 19.5 | 1.56 | 10 | 8.1 | CH₂ | (—) |
| LIPO/17AAG/ | | | | | | | | Cl₂/ | |
| ST_4 | | | | | | | | HO₂ | |
| PLGA/PVA/ | 80.0 | 200.0 | 19.5 | 9.75 | 0.78 | 11.5 | 9.5 | CH₂ | (—) |
| LIPO/17AAG/ | | | | | | | | Cl₂/ | |
| ST_5 | | | | | | | | PBS | |
| | | | | | | | | 7.4 | |
| PLGA/PVA/ | 80.0 | 200.0 | 19.5 | 9.75 | 0.78 | 10 | — | CH₂ | Glucose |
| LIPO/17AAG/ | | | | | | | | Cl₂/ | |
| 3G | | | | | | | | PBS | |
| | | | | | | | | 7.4 | |
| PLGA/PVA/ | 80.0 | 200.0 | 19.5 | 9.75 | 0.78 | 10 | — | CH₂ | Non-ionic |
| LIPO/17AAG/ | | | | | | | | Cl₂/ | surfactant |
| 1T | | | | | | | | PBS | |
| | | | | | | | | 7.4 | |
| PLGA/PVA/ | 80.0 | 200.0 | 19.5 | 9.75 | 0.78 | — | 10 | CH₂ | Glucose |
| LIPO/ST/1G | | | | | | | | Cl₂/ | |
| | | | | | | | | PBS | |
| | | | | | | | | 7.4 | |
| PLGA/PVA/ | 80.0 | 200.0 | 19.5 | 9.75 | 0.78 | — | 10 | CH₂ | Non-ionic |
| LIPO/ST/4T | | | | | | | | Cl₂/ | surfactant |
| | | | | | | | | PBS | |
| | | | | | | | | 7.4 | |
| PLGA/PVA/ | 80.0 | 200.0 | 19.5 | 9.75 | 0.78 | 8.4 | 1.7 | CH₂ | Glucose |
| LIPO/17AAG/ | | | | | | | | Cl₂/ | |
| ST/1G | | | | | | | | PBS | |
| | | | | | | | | 7.4 | |
| PLGA/PVA/ | 80.0 | 200.0 | 19.5 | 9.75 | 0.78 | 8.4 | 1.7 | CH₂ | Non-ionic |
| LIPO/17AAG/ | | | | | | | | Cl₂/ | surfactant |
| ST/1T | | | | | | | | PBS | |
| | | | | | | | | 7.4 | |
| PLGA/PVA/ | 80.0 | 200.0 | 19.5 | 9.75 | 0.78 | 10 | 2.0 | CH₂ | Glucose |
| LIPO/17AAG/ | | | | | | | | Cl₂/ | |
| ST/2G | | | | | | | | PBS | |
| | | | | | | | | 7.4 | |
| PLGA/PVA/ | 80.0 | 200.0 | 19.5 | 9.75 | 0.78 | 10 | 2.0 | CH₂ | Non-ionic |
| LIPO/17AAG/ | | | | | | | | Cl₂/ | surfactant |
| ST/2T | | | | | | | | PBS | |
| | | | | | | | | 7.4 | |
| PLGA/PVA/ | 80.0 | 200.0 | 19.5 | 9.75 | 0.78 | 10 | 1.0 | CH₂ | Glucose |
| LIPO/17AAG/ | | | | | | | | Cl₂/ | |
| ST/3G | | | | | | | | PBS | |
| | | | | | | | | 7.4 | |
| PLGA/PVA/ | 80.0 | 200.0 | 19.5 | 9.75 | 0.78 | 10 | 1.0 | CH₂ | Non-ionic |
| LIPO/17AAG/ | | | | | | | | Cl₂/ | surfactant |
| ST/3T | | | | | | | | PBS | |
| | | | | | | | | 7.4 | |
| PLGA/PVA/ | 80.0 | 200.0 | 19.5 | 9.75 | 0.78 | 5 | 1.0 | CH₂ | Glucose |
| LIPO/17AAG/ | | | | | | | | Cl₂/ | |
| ST/4G | | | | | | | | PBS | |
| | | | | | | | | 7.4 | |
| PLGA/PVA/ | 80.0 | 200.0 | 19.5 | 9.75 | 0.78 | 5 | 1.0 | CH₂ | Non-ionic |
| LIPO/17AAG/ | | | | | | | | Cl₂/ | surfactant |
| ST/4T | | | | | | | | PBS | |
| | | | | | | | | 7.4 | |

TABLE 3
Physicochemical parameters of PLGA/PVA/LIPO nanoparticles - size (hydrodynamic
radius, HR), polydispersity index (PDI), drug encapsulation rates (% EE)
and the ratio of C/C_{17AAGST} concentrations expressed in mg/ml for
nanoparticles loaded with more than one drug.

| | Size/HR | | | | % | % | C/ |
|---|---|---|---|---|---|---|---|
| L.p. | [nm] | SD | PDI | SD | EE17AAG | EEST | C_{17AAGST} |
| PLGA/PVA/LIPO | 387.9 | 9.32 | 0.043 | 0.003 | — | — | — |
| PLGA/PVA/LIPO/ | 223.7 | 3.21 | 0.087 | 0.011 | 55 | 75 | 0.91 |
| 17AAG/ST_1 | | | | | | | |
| PLGA/PVA/LIPO/ | 209.4 | 3.23 | 0.126 | 0.016 | 75 | 79 | 1.2 |
| 17AAG/ST_2 | | | | | | | |
| PLGA/PVA/LIPO/ | 214.6 | 1.25 | 0.097 | 0.023 | 32 | 63.5 | 0.62 |
| 17AAG/ST_3 | | | | | | | |
| PLGA/PVA/LIPO/ | 264.1 | 0.96 | 0.172 | 0.024 | 74.4 | 75 | 1.2 |
| 17AAG/ST_4 | | | | | | | |
| PLGA/PVA/LIPO/ | 237.0 | 2.56 | 0.030 | 0.010 | 79.2 | 74 | 1.3 |
| 17AAG/ST_5 | | | | | | | |
| PLGA/PVA/LIPO/ | 227.2 | 2.40 | 0.085 | 0.021 | 56 | — | — |
| 17AAG/3G | | | | | | | |
| PLGA/PVA/LIPO/ | 226.1 | 0.76 | 0.108 | 0.012 | 69.4 | — | — |
| 17AAG/1T | | | | | | | |
| PLGA/PVA/LIPO/ | 298.4 | 4.34 | 0.141 | 0.003 | — | 75.5 | — |
| ST/1G | | | | | | | |
| PLGA/PVA/LIPO/ | 298.9 | 3.40 | 0.122 | 0.016 | — | 79.5 | — |
| ST/4T | | | | | | | |
| PLGA/PVA/LIPO/ | 220.2 | 0.65 | 0.076 | 0.031 | 53.4 | 61.5 | 4.3 |
| 17AAG/ST/1G | | | | | | | |
| PLGA/PVA/LIPO/ | 225.6 | 3.03 | 0.033 | 0.017 | 60 | 84 | 3.5 |
| 17AAG/ST/1T | | | | | | | |
| PLGA/PVA/LIPO/ | 245.2 | 3.5 | 0.166 | 0.0004 | 61.7 | 51.5 | 6.0 |
| 17AAG/ST/2G | | | | | | | |
| PLGA/PVA/LIPO/ | 231.4 | 1.93 | 0.038 | 0.03 | 72.2 | 74.6 | 4.8 |
| 17AAG/ST/2T | | | | | | | |
| PLGA/PVA/LIPO/ | 220.8 | 0.18 | 0.068 | 0.013 | 46.2 | 63 | 7.3 |
| 17AAG/ST/3G | | | | | | | |
| PLGA/PVA/LIPO/ | 217.5 | 2.48 | 0.048 | 0.025 | 71 | 75 | 9.5 |
| 17AAG/ST/3T | | | | | | | |
| PLGA/PVA/LIPO/ | 230.3 | 0.66 | 0.108 | 0.026 | 45.6 | 58.5 | 3.9 |
| 17AAG/ST/4G | | | | | | | |
| PLGA/PVA/LIPO/ | 216.7 | 2.2 | 0.019 | 0.015 | 60.2 | 75 | 4 |
| 17AAG/ST/4T | | | | | | | |

### Example I Nanoparticles PLGA/PVA/LIPO/17AAG/3G

200 mg of PVA, 80 mg of PLGA and 10.0 mg of 17AAG were weighed, according to Table 2, for the PLGA/PVA/LIPO/17AAG/1G nanoparticle. PVA was dissolved in 10 ml of PBS buffer 7.4 using ultrasound—about 1 h, until the PVA crystals were completely dissolved, then left in the refrigerator overnight. PLGA was dissolved in 5 ml of dichloromethane and ultrasounded for 5 s. 17AAG was then dissolved in the PLGA solution. It was cooled to 4° C. (60 minutes in the fridge). The dichloromethane solution with PLGA and 17AAG was then added to a large glass vessel (50 ml bottle) followed by the PVA aqueous solution, again cooled in the fridge for 30 minutes. After this time, the whole mixture was stirred until an emulsion was obtained using an ultrasonic probe for 1 minute, amplitude 80%. The ultrasonic probe was immersed at the interface between the two phases about 5 mm above the interface. The resulting emulsion was poured into a 100 ml crystalliser with a stirring element making sure that all the emulsion was transferred into the crystalliser. The bottle was washed with 2 ml of PBS 7.4 (the solution after washing was placed in the crystalliser). The whole was left on a magnetic stirrer for 12 h (400 RPM, 37° C.) until the dichloromethane was completely evaporated. The solution was made up to 15 mL with PBS buffer pH 7.4. 1 mL each of PLGA/PVA nanoparticles loaded with 17AAG (unpurified) and then 200 uL of liposomes (DPPC/Cholesterol/DSPE_PEG(2000)NH2 (1/0.5/0.04) 10 mg/ml) synthesised in 0.9% NaCl were added to glass bottles (10 mL) with a stirring element. Samples were heated in a water bath for 4 h, 45° C., stirring with a magnetic stirrer with heating (400 RPM). It was then left in the refrigerator for 12 h. 0.5 ml of the sample was left for determination of the % EE encapsulation rate by chromatography on a Sephadex G-25 packed column. The rest of the nanoparticles were purified using a high-speed centrifuge (15000 RPM, 4 min), washing twice with PBS 7.4 buffer. After each addition of PBS 7.4, the nanoparticles were redispersed using ultrasound for 5 seconds. The Zeta potential of the nanoparticles was measured and is shown in FIG. 1.1. After the last addition of PBS 7.4 buffer, 20 uL of glucose solution (100 mg/ml) was added to the nanoparticles for every 1 ml of nanoparticles. PLGA/PVA/LIPO/17AAG/3G nanoparticles were thus obtained. The size and polydispersity coefficient of PLGA/PVA/LIPO/17AAG/3G nanoparticles were then determined using the DLS method. Subsequently, the nanoparticles were sterilised using sterile PTFE-filled hydrophilic syringe filters with a pore diameter of 0.45 μm and the size and PDI were again determined. FIG. 1.2 shows the distribution of hydrodynamic radii for the PLGA/PVA/LIPO/17AAG/3G nanoparticle together with the PDI polydispersity index after the sterilisation process.

The concentration of loaded drugs and % EE were determined by UV-Vis spectroscopy against a calibration curve at 333 nm wavelengths. The nanoparticle sample was dissolved in DMSO with hydrochloric acid according to the ratio: 50 uL of sample, 50 uL of 0.3 M HCl, 50 uL of PBS 7.4 and 850 uL of DMSO and a spectrum was taken. The spectrum is included in FIG. 1.3A and FIG. 1.3B

In addition, the antiproliferative activity of PLGA/PVA/LIPO/17AAG/3G nanoparticles was assessed in comparison to the free drug. In vitro cultured lung cancer and glioma tumour cells were seeded into sterile 384-well plates at 1,000 cells per well. After a 24-hour incubation (37° C., 5% CO2, 95% humidity), cells were treated with 17AAG solutions or PLGA/PVA/LIPO/17AAG/3G nanoparticles in at least eight different concentrations. Cells were then incubated for 72 hours (37° C., 5% CO2, 95% humidity) and cell growth inhibition was assessed using the SRB assay. Briefly, the protein in each test well was precipitated with 20% trichloroacetic acid solution for 1 hour, washed and labelled using 0.4% sulforhodamine B solution in 1% acetic acid (30 minutes). After washing off the excess dye with 1% acetic acid solution, the protein-bound dye was dissolved in 10 mM TRIS buffer solution (30 minutes) and the absorbance level was read using a spectrophotometer at 540 nm. Proliferation inhibition was calculated using the formula:

$$\% ZahInh = ( ( \frac{{A}_{p} - {A}_{m}}{{A}_{k} - {A}_{m}} ) \times 100 ) - 100$$

### Where:

% ZahInh—inhibition of proliferation
Aₘ—absorbance of wells without cells (medium)
Aₖ—absorbance of control wells (cells treated with pure culture medium)
Aₚ—absorbance of wells treated with compounds

The results are shown in FIGS. 1.4-1.11

Conclusions: The drug 17-AAG after loading into the engineered nanocarriers showed antiproliferative activity at least comparable to that shown by the free drug. A significant increase in the activity of compound 17AAG after loading into the nanocarrier was observed for a significant proportion of the lines, particularly for lower drug concentrations, indicating the possibility of lowering the therapeutic doses of PLGA/PVA/LIPO/17AAG/3G relative to free 17AAG while maintaining the required antitumour activity.

### Example II. PLGA/PVA/LIPO/ST/4T Nanoparticles

Weighed 200 mg PVA, 80 mg PLGA, according to Table 2, for the PLGA/PVA/LIPO/ST/$T nanoparticle. PVA was dissolved in 10 ml of PBS buffer 7.4 using ultracentrifugation—about 1 h, until the PVA crystals were completely dissolved, then left in the refrigerator overnight. PLGA was dissolved in 5 ml of dichloromethane and ultrasonicated for 5 s. Then 10 mg of Staurosporine was weighed and dissolved in the PLGA solution. It was cooled to 4° C. (30 minutes in the refrigerator). Next, a solution of dichloromethane with PLGA and Staurosporine, and an aqueous solution of PVA was added to a large glass vessel (100 ml bottle), again cooled in the fridge for 30 minutes. After this time, the whole was stirred until an emulsion was obtained using an ultrasonic probe for 2 minutes, amplitude 80%. The ultrasonic probe was immersed at the interface between the two phases about 5 mm above the interface. The resulting emulsion was poured into a 50 ml crystalliser with a mixing element making sure that all the emulsion was transferred into the crystalliser. The bottle was washed with 2 ml of PBS 7.4 (the solution after washing was placed in the crystalliser). The whole was left on a magnetic stirrer for 2 h (400 RPM, 37° C.) until the dichloromethane was completely evaporated. The solution was made up to 15 mL with PBS buffer pH7.4. 1 mL each of PLGA/PVA nanoparticles loaded with Staurosporine (unpurified) and then 200 uL of liposomes (DPPC/Cholesterol/DSPE_PEG(2000)NH2 (1/0.5/0.04) 10 mg/ml) synthesised in 0.9% NaCl were added to glass bottles (10 mL) with a stirring element. Samples were heated in a water bath for 2 h, 60° C., stirring with a magnetic stirrer with heating (400 RPM). It was then left in the refrigerator for 14 h. 0.5 ml of the sample was left for determination of the % EE encapsulation rate by chromatography on a Sephadex G-25 packed column. The rest of the nanoparticles were purified using a high-speed centrifuge (15000 RPM, 4 min), washing twice with PBS 7.4 buffer. After each addition of PBS 7.4, the nanoparticles were redispersed using ultrasound for 5 seconds. The Zeta potential of the nanoparticles was measured, shown in FIG. 2.1. After the last addition of PBS 7.4 buffer, 20 uL of a 40% solution of a of non-ionic surfactant for every 1 mL of nanoparticles. PLGA/PVA/LIPO/ST/4T nanoparticles were thus obtained. The size and polydispersity coefficient of the PLGA/PVA/LIPO/ST/4T nanoparticles were then determined using the DLS method. Subsequently, the nanoparticles were sterilised using sterile PTFE-filled hydrophilic syringe filters with a pore diameter of 0.45 μm and the size and PDI were again determined. FIG. 2.2 shows the distribution of hydrodynamic radii for the PLGA/PVA/LIPO/ST/4T nanoparticle together with the PDI polydispersity index after the sterilisation process.

The concentration of loaded staurosporine and % EE were determined using:
UV-Vis spectroscopy against a calibration curve at 372, 294 and 353 nm. The nanoparticle sample was dissolved in DMSO with hydrochloric acid according to the ratio: 25 uL of sample, 50 uL of 0.3M HCl, 75 uL of PBS 7.4 and 850 uL of DMSO and a spectrum was taken as shown in FIG. 2.3.
chromatographic method by purifying a 0.5 ml sample of the final PLGA/PVA/LIPO/ST/4T nanoparticles. Purification was carried out on Sephadex G-25 packed chromatography columns. After depositing a 0.5 ml sample of unpurified PLGA/PVA/LIPO/ST/4T nanoparticles on a Sephadex G-25 packed column, 8 fractions of 1 ml were collected with PBS buffer pH 7.4. The content of each fraction was controlled by UV-Vis spectroscopy. Fractions 3-5 containing PLGA/PVA/LIPO/ST/4T nanoparticles were pooled together and the drug concentration of the nanoparticles was determined by UV-Vis spectroscopy against a calibration curve. The PLGA/PVA/LIPO/ST/4T sample was dissolved in DMSO with hydrochloric acid according to the ratio of 50 uL sample, 50 uL 0.3M HCl, 50 uL PBS 7.4 and 850 uL DMSO. Subsequently, unbound Staurosporine was eluted from the column by washing the chromatography column with 25 ml of a 50% ethanol solution. The concentration of unbound staurosporine was determined against a calibration curve of Staurosporine in 50% ethanol at 372, 294 and 353 nm.

The results of the % EE calculations are shown in summary Table 3.

In addition, the antiproliferative activity of PLGA/PVA/LIPO/ST/4T nanoparticles was assessed in comparison to free Staurosporine. In vitro cultured lung cancer and glioma tumour cells were seeded in sterile 384-well plates at 1,000 cells per well. After a 24-hour incubation (37° C., 5% CO2, 95% humidity), cells were treated with Staurosporine solutions or PLGA/PVA/LIPO/ST/4T nanoparticles in at least 8 different concentrations. Cells were then incubated for 72 hours (37° C., 5% CO2, 95% humidity) and cell growth inhibition was assessed using the SRB assay. Briefly, the protein in each test well was precipitated with 20% trichloroacetic acid solution for 1 hour, washed and labelled using 0.4% sulforhodamine B solution in 1% acetic acid (30 minutes). After washing off the excess dye with 1% acetic acid solution, the protein-bound dye was dissolved in 10 mM TRIS buffer solution (30 minutes) and the absorbance level was read using a spectrophotometer at 540 nm. Proliferation inhibition was calculated using the formula:

$$\% ZahInh = ( ( \frac{{A}_{p} - {A}_{m}}{{A}_{k} - {A}_{m}} ) \times 100 ) - 100$$

### Where:

% ZahInh—inhibition of proliferation
Aₘ—absorbance of wells without cells (medium)
Aₖ—absorbance of control wells (cells treated with pure culture medium)
Aₚ—absorbance of wells treated with compounds

The results are shown in FIGS. 2.3-2.11

Conclusions: Staurosporine after loading into the engineered nanocarriers showed antiproliferative activity at least comparable to that shown by free Staurosporine. For some of the lines, a significant increase in the activity of compound 17AAG was observed after loading into the nanocarrier indicating the possibility of lowering the therapeutic doses of PLGA/PVA/LIPO/ST/4T relative to free Staurosporine while maintaining the antitumour activity.

### Example III. PLGA/PVA/LIPO/17AAG/ST Polymeric Nanoparticle Loaded with Staurosporine and 17AAG Coated with DPPC/Cholestrol/DSPE_PEG(2000)NH2 Lipid Bilayer

200 mg of PVA, 100 mg of PLGA, 9.5 mg of Staurosporine and 11.5 mg of 17AAG were weighed according to Table 2, for the PLGA/PVA/LIPO/17AAG/ST_2 nanoparticle. The PVA was dissolved in 10 ml of PBS buffer 7.4 using ultrasound—about 1 h, until the PVA crystals were completely dissolved, then left in the refrigerator overnight. PLGA, Staurosporine and 17AAG were dissolved in 5 ml of dichloromethane and ultrasonicated for 5 s. It was cooled to 4° C. (30 min in the fridge). A solution of dichloromethane with PLGA, Staurosporine and 17AAG was then added to a large glass vessel (50 ml bottle) followed by an aqueous solution of PVA, again cooled in the fridge for 30 minutes. After this time, the whole mixture was stirred until an emulsion was obtained using an ultrasonic probe for 1.5 minutes, amplitude 80%. The ultrasonic probe was immersed at the boundary between the two phases about 5 mm above the phase boundary. After sonification, a homogeneous emulsion was obtained. The emulsion was poured into a 50 ml crystalliser with a mixing element making sure that all the emulsion was transferred into the crystalliser. The bottle was washed with 2 ml of PBS 7.4 (the solution was placed in the crystalliser after washing). The whole was left on a magnetic stirrer for 2 h (400 RPM, 37° C.) until the dichloromethane was completely evaporated. The solution was then made up to 15 mL with PBS buffer pH7.4. 1 mL each of PLGA/PVA nanoparticles loaded with Staurosporine and 17AAG (unpurified) and then 200 uL of liposomes (DPPC/Cholesterol/DSPE_PEG(2000)NH2 (1/0.5/0.04) 10 mg/ml) synthesised in 0.9% NaCl were added to 4 glass bottles (10 mL) with a stirring element. Samples were heated in a water bath for 3 h, 50° C., stirring with a magnetic stirrer with heating (400 RPM). They were then left in the refrigerator for 16 h. 0.5 ml of the sample was left for determination of the % EE encapsulation rate by chromatography on a chromatography column. The rest of the nanoparticles were purified using a high-speed centrifuge (15000 RPM, 4 min), washing twice with PBS 7.4 buffer. After each addition of PBS 7.4, the nanoparticles were redispersed using ultrasound for 5 seconds. PLGA/PVA/LIPO/17AAG/ST_2 nanoparticles were thus obtained. The size and polydispersity coefficient of the PLGA/PVA/LIPO/17AAG/ST_2 nanoparticles were then determined using the DPL method. Subsequently, the nanoparticles were sterilised using sterile PTFE-filled hydrophilic syringe filters with a pore diameter of 0.45 μm and the size, PDI and Zeta potential of the nanoparticles were again determined. FIG. 3.1A shows the distribution of hydrodynamic radii for the PLGA/PVA/LIPO/17AAG/ST_2 nanoparticle together with the PDI polydispersity index after the sterilisation process. FIG. 3.1B shows the Zeta potential distribution.

The concentration of loaded drugs and % EE were determined using:
UV-Vis spectroscopy against a calibration curve at 372 nm and 333 nm, using the principle of absorbance additivity. The nanoparticle sample was dissolved in DMSO with hydrochloric acid according to the ratio: 50 uL of sample, 50 uL of 0.3M HCl, 50 uL of PBS 7.4 and 850 uL of DMSO and a spectrum was taken.
chromatographic method, purifying 0.5 ml samples of the final PLGA/PVA/LIPO/17AAG/ST_2 nanoparticles. Purification was performed on Sephadex columns with drug separation. After application of a 0.5 ml sample of unpurified PLGA/PVA/LIPO/17AAG/ST_2 nanoparticles onto a Sephadex G-25 packed column, 8 fractions of 1 ml were collected with PBS buffer pH 7.4. The content of each fraction was controlled by UV-Vis spectroscopy. Fractions 4-7 containing PLGA/PVA/17AAG/ST nanoparticles were pooled together and drug concentrations on the nanoparticles were determined by UV-Vis spectroscopy against a calibration curve using the principle of additivity of absorbance by dissolving the sample from fractions 4-7 in DMSO with hydrochloric acid according to the ratio: 50 uL of sample, 50 uL of 0.3M HCl, 50 uL of PBS 7.4 and 850 uL of DMSO. Subsequently, unbound 17AAG from the column was eluted from the column by washing the chromatography column with 25 ml of PBS buffer pH 7.4 and the concentration of unbound 17AAG relative to the 17AAG calibration curve in PBS buffer at 333 nm was determined. Unbound Staurosporine was then eluted from the column by washing the column with 25 ml of Ethanol:Water solution (1:1). The concentration of free Staurosporine was determined by UV-Vis spectroscopy against a calibration curve at 372, 294 and 353 nm. The results are shown in summary Table 3.

The stability of PLGA/PVA/LIPO/17AAG/ST nanoparticles was tested over a temperature range of 25 to 45° C. at endosomal/lysosomal pH (pH 5, pH 6) and pH characteristic of the bloodstream (pH 7.4) 24 hours after synthesis and after seven days of storage at 4° C. for pH 7.4.

FIGS. 3.2 and 3.3 show the dependence of the hydrodynamic diameter (HR) of PLGA/PVA/17AAG/ST/Lipo_2 nanoparticles and the PDI coefficient in the temperature range from 25° C. to 45° C. in pH buffers characteristic of endosomes and lysosomes (pH5, pH 6) and neutral pH (pH 7.4) for nanoparticles 24 h after synthesis and 7 days after synthesis (at pH 7.4).

The absence of significant changes in hydrodynamic diameter from PDI for the temperature range from 25° C. to 45° C. at pH 5, 6 and 7.4 demonstrates the stability of PLGA/PVA/17AAG/ST/Lipo_2 nanoparticles over the tested temperature range, pH and after time.

For PLGA/PVA/LIPO/17AAG/ST_2 nanoparticles, FIG. 3.4 shows the drug release kinetics in PBS buffer at pH 7.4 compared to purified PLGA/PVA nanoparticles loaded with 17AAG and Staurosporine (ST) but not subjected to lipid envelope coating of the nanoparticles

In the case of PLGA/PVA nanoparticles loaded with 17AAG and ST not subjected to lipid bilayer coating, 17AAG release as early as 5 h reached a value close to 30%, while after 24 h it reached 45%, whereas lipid-coated PLGA/PVA/LIPO/17AAG/ST nanoparticles are characterised by a lack of drug release and a gradual release of 17AAG with an average value: 10% after 5 h, 25% after 24 h and 30% after 48 h. The value of the mean ST release after time was independent of the presence of the lipid-polymer envelope.

The effect of the protein on the amount of drug release at 37° C. over time was further investigated. FIG. 3.5 shows an example of the release profile of 17AAG and ST in PBS buffer in the presence of 5% BSA recorded at 37° C.

In the presence of %5 BSA, PLGA/PVA/LIPO/17AAG/ST_2 nanoparticles show an average release of both 17AAG and ST drugs of 30% after both 24 h and 48 h. After 72 h, a release of both drugs of 55% is observed. For the PLGA/PVA/17AAG/ST/Lipo_2 nanoparticles, no sudden drug release from the nanoparticle was observed until 48 h, the drugs releasing gradually.

The stability of nanoparticle structures was investigated using the PLGA/PVA/LIPO/17AAG/ST_2 nanoparticle as an example in PBS buffer 7.4 with the addition of 5% bovine BSA albumin to exclude nanoparticle aggregation in in vitro and in vivo studies. Albumins are the most abundant proteins in the bloodstream and are used as an additive to the cell medium in in vitro studies. The size distributions of hydrodynamic radii for PLGA/PVA/LIPO/17AAG/ST_2 nanoparticles after the addition of 5% bovine albumin after 1 h, 2 h, 3 h, 4 h, 24 h, 28 h, 96 h and 120 h are shown in FIGS. 3.6-3.13. In addition, the same tests were performed for PLGA/PVA nanoparticles but not coated with lipid bilayer. The hydrodynamic radius distributions for PLGA/PVA liposomes before and after the addition of a 10% BSA solution by volume (1:1) at T=0 at 25° C. and after one hour of incubation at 37° C. are shown in FIGS. 3.14-3.16.

After the addition of 5% BSA to the PLGA/PVA/LIPO/17AAG/ST_2 nanoparticle solution, a slight increase in the mean hydrodynamic radius of the nanoparticle was observed, indicating the interaction of the nanoparticle with bovine albumin and the deposition of a small amount of protein on the nanoparticle surface. No appearance of larger nanostructures was observed, indicating the stability of the nanoparticles in the presence of plasma protein. Only after 120 h of incubation at 37° C. in the presence of BSA was a signal from a small number of larger structures above 1 um observed, indicating an initial loss of stability by the nanoparticles. The above results indicate that PLGA/PVA/LIPO/17AAG/ST_2 nanoparticles are stable in the presence of proteins for up to 120 h. In contrast to lipid-coated nanoparticles, PLGA/PVA nanoparticles lacking the lipid coating aggregate in 5% BSA, indicating their instability in the presence of plasma proteins.

Stability studies were performed on PLGA/PVA/LIPO/17AAG/ST_2 nanoparticles during storage in three different solutions: in 2 mg/ml glucose solution, in 0.8% Tween 20 solution and in 0.1 M PBS buffer, pH 7.4. Measurements were performed at weekly intervals for 4 consecutive weeks. All collected fractions and test solutions were stored in a refrigerator (temp. 4° C.-7° C.) for subsequent measurements.

Each time, drug concentrations (staurosporine and 17-AAG) were determined for each of the 3 test samples. For this purpose, solutions containing 50 μl of the test sample, 50 μl of 0.3 M HCl solution, 50 μl of PBS buffer pH 7.4 and 850 μl of DMSO (20-fold dilution) were prepared in acrylic cuvettes. The absorbance of the thus prepared solutions was then recorded using a UV-Vis spectrophotometer. Drug concentrations were determined relative to the calibration curves using the principle of additivity of absorbance.

In the next step, repeated analogously for 4 weeks for each of the three samples tested, hydrodynamic diameters and polydispersity indices (PDI) were determined using the dynamic light scattering (DLS) technique. The data are summarised in Table 4.

TABLE 4
Values of hydrodynamic diameters and PDI polydispersity
coefficients for PLGA/PVA/LIPO/17AAG/ST_2 nanoparticles
measured after 0, 7, 14, 21, 28 days of storage at 4°
C.-7° C. with the addition of stabilising agents.

| | | | | |
|---|---|---|---|---|
| Time | Hydrodynamic | | | |
| [days] | diameter [nm] | SD | PDI | SD |

PLGA/PVA/LIPO/17AAG/ST_2 in glucose solution 2 mg/ml

| | | | | |
|---|---|---|---|---|
| 0 | 305.4 | 60.33 | 0.197 | 0.010 |
| 7 | 293.7 | 27.88 | 0.229 | 0.030 |
| 14 | 377.5 | 25.50 | 0.210 | 0.026 |
| 21 | 417.3 | 51.48 | 0.205 | 0.026 |
| 28 | 370.4 | 69.20 | 0.223 | 0.024 |

PLGA/PVA/LIPO/17AAG/ST_2 in 0.8% Tween 20 solution

| | | | | |
|---|---|---|---|---|
| 0 | 254.2 | 19.46 | 0.141 | 0.025 |
| 7 | 234.3 | 4.14 | 0.229 | 0.030 |
| 14 | 265.2 | 14.34 | 0.126 | 0.019 |
| 21 | 249.7 | 6.57 | 0.177 | 0.027 |
| 28 | 256.9 | 7.15 | 0.087 | 0.002 |

PLGA/PVA/LIPO/17AAG/ST_2 in 0.1M PBS buffer 7.4

| | | | | |
|---|---|---|---|---|
| 0 | 255.60 | 31.46 | 0.189 | 0.025 |
| 7 | 307.20 | 46.80 | 0.183 | 0.019 |
| 14 | 272.70 | 24.56 | 0.196 | 0.043 |
| 21 | 270.80 | 13.02 | 0.158 | 0.026 |
| 28 | 285.40 | 4.79 | 0.181 | 0.020 |

In addition, drug leakage from the nanoparticles was controlled using chromatographic methods on a Sephadex G-50 bed.

A 200 μl sample was applied to the previously prepared column and 4 fractions of 1 ml, 4 fractions of 0.5 ml and 2 fractions of 1 ml were collected successively into acrylic cuvettes. After the 10 fractions had been collected, their absorbance was examined in the wavelength range 220-800 nm using a UV-Vis spectrophotometer. Based on the UV-Vis spectra, the liposomal fraction was detected and pooled together. The remaining fraction containing the free drug (17AAG) unbound to the nanoparticles was transferred to a volumetric flask and further eluted from the column with PBS buffer, pH 7.4, up to a volume of 25 ml. Subsequently, the column was washed with a 50% ethanol solution to elute the Staurosporine unbound nanoparticles from the column. Depending on the sample, between 25 ml and 35 ml of solution was collected. The concentration of free Staurosporine was then determined by UV-Vis spectrophotometry against a calibration curve.

The drug concentration of the liposomal fractions was determined by UV_Vis spectrophotometry in analogy with the total drug concentration. Solutions containing 100 μl of the liposomal fraction of the test sample, 50 μl of 0.3 M HCl solution and 850 μl of DMSO were prepared in acrylic cuvettes. The solutions were mixed using an automatic pipette, after which their absorbance was measured and drug concentrations were determined relative to the calibration curves using the principle of additivity of absorbance.

The drug content of PLGA/PVA/LIPO/17AAG/ST_2 nanoparticles stored after 0, 7, 14, 21 and 28 days is shown in FIG. 3.17 for staurosporine and FIG. 3.18 for 17AAG.

PLGA/PVA/LIPO/17AAG/ST_2 nanoparticles have a relatively low, acceptable rate of drug leakage from the nanoparticle of up to 15% within 4 weeks of storage at 4° C.-7° C. The addition of stabilising substances such as glucose or non-ionic surfactants eliminates drug leakage from the nanoparticle.

The stability of the physical parameters was checked 4 months after synthesis. The data are summarised in Table 4.

TABLE 4
Physical parameters of PLGA/PVA/LIPO nanoparticles
after synthesis and 4 months after synthesis.

| PLGA/ | | | | | | | | |
|---|---|---|---|---|---|---|---|---|
| PVA/ | After synthesis | | | | 4 months after synthesis | | | |
| LIPO/ | Z- | ±Z- | | | Z- | ±Z- | | |
| 17AAG/ | Average | average | | | Average | average | | |
| ST/ | [nm] | [nm]. | PDI | ±PDI | [nm] | [nm]. | PDI | ±PDI |
| 1G | 220.20 | 0.65 | 0.08 | 0.03 | 223.00 | 4.94 | 0.11 | 0.04 |
| 1T | 225.60 | 3.03 | 0.03 | 0.02 | 213.30 | 3.11 | 0.04 | 0.01 |
| 2G | 245.20 | 3.50 | 0.17 | 0.00 | 224.30 | 3.37 | 0.02 | 0.01 |
| 2T | 231.40 | 1.93 | 0.04 | 0.03 | 217.60 | 4.62 | 0.02 | 0.02 |
| 3G | 220.80 | 0.18 | 0.07 | 0.01 | 232.30 | 9.98 | 0.19 | 0.04 |
| 3T | 217.50 | 2.48 | 0.05 | 0.03 | 205.60 | 4.39 | 0.01 | 0.01 |
| 4G | 230.30 | 0.66 | 0.11 | 0.03 | 228.80 | 5.14 | 0.16 | 0.07 |
| 4T | 216.70 | 2.20 | 0.02 | 0.02 | 207.90 | 0.03 | 0.03 | 0.01 |

Additionally, the anti proliferative activity of PLGA/PVA/LIPO/17AAG/ST_2 nanoparticles was assessed in comparison to the free drug mixture 17AAG+Staurosporine. In vitro cultured lung cancer and glioma tumour cells were seeded into sterile 384-well plates at 1,000 cells per well. After a 24-hour incubation (37° C., 5% CO2, 95% humidity), the cells were treated with 17AAG+Staurosporine drug mixture solutions or PLGA/PVA/LIPO/17AAG/ST_2 nanoparticles at at least 8 different concentrations relative to the more anticancer active drug Staurosporine. Cells were then incubated for 72 hours (37° C., 5% CO2, 95% humidity) and cell growth inhibition was assessed using the SRB assay. Briefly, the protein in each test well was precipitated with 20% trichloroacetic acid solution for 1 hour, washed and labelled using 0.4% sulforhodamine B solution in 1% acetic acid (30 minutes). After washing off the excess dye with 1% acetic acid solution, the protein-bound dye was dissolved in 10 mM TRIS buffer solution (30 minutes) and the absorbance level was read using a spectrophotometer at 540 nm. Proliferation inhibition was calculated using the formula:

$$\% ZahInh = ( ( \frac{{A}_{p} - {A}_{m}}{{A}_{k} - {A}_{m}} ) \times 100 ) - 100$$

### Where:

% ZahInh—inhibition of proliferation
Aₘ—absorbance of wells without cells (medium)
Aₖ—absorbance of control wells (cells treated with pure culture medium)
Aₚ—absorbance of wells treated with compounds

The results are shown in FIGS. 3.19-3.2

Conclusions: The drug mixture 17AAG+Staurosporine when loaded into the developed nanocarriers showed antiproliferative activity significantly higher than the free drug mixture 17AAG+Staurosporine. This offers the possibility to significantly reduce the therapeutic doses of PLGA/PVA/LIPO/17AAG/ST_2 compared to free Staurosporine while maintaining high antitumour activity.

1. Stable polymer-lipid nanoparticle for drug binding and delivery, especially with anticancer properties, characterised by being composed of a core containing polylactic-co-glycolic acid (PLGA) in an amount of 16.1 to 30.3% by weight and polyvinyl alcohol (PVA) in an amount of 60.6 to 75.5% by weight, and a core shell containing a mixture of lipids: 1,2-dipalmitoyl-sn-glycero-3-phosphocholine (DDPC) in an amount ranging from 3.66% to 12.6% by weight, cholesterol in an amount ranging from 1.85 to 6.29% by weight and the ammonium salt 1,2-distearoyl-sn-glycero-3-phosphoethanolamine-N-[amino(polyethylene glycol)-2000 (DSPE-PEG(2000)NH2] in an amount ranging from 0.15 to 0.5% by weight.

2. The nanoparticle according to claim 1, characterised in that the PLGA is a 50:50 copolymer with a mass Mw of 24 000-38 000 g/mol.

3. The nanoparticle according to claim 1, characterised in that the PVA has a mass Mw of 30 000-70 000 g/mol.

4. A method for the manufacture of stable polymer-lipid nanoparticles that carry drugs, in particular with anticancer properties, characterised by the following
in the first step, the core is obtained by preparing a clear aqueous solution of PVA at concentrations of 15 mg/ml to 20 mg/ml, followed by a solution of PLGA in dichloromethane at concentrations of 10 mg/ml to 20 mg/ml, at least one drug is then weighed out in the amount needed to make solutions with a concentration range of 0.43 mM to 4.30 mM and dissolved in the PLGA solution, after which both solutions are cooled to between 4° C. and 7° C. and then mixed in a volume ratio of 1:2, where one part of PLGA solution in dichloromethane with the drug is equal to two parts of aqueous PVA solution, and again cooled to 4° C. to 7° C. and the whole is mixed to form an emulsion; the emulsion is poured into a crystalliser with a stirring element and the residue is washed with PBS buffer pH 7.4 or water at a volume ratio of the rinse solution (PBS or water) to the reaction mixture of 1:7.5 to 1:10 and also placed in the crystalliser, after which the dichloromethane is completely evaporated, the volume is then replenished with PBS 7.4 solution or water until a final PLGA concentration of 3.33 mg/ml to 6.67 mg/ml is obtained,
in a second step, the previously formed core is covered with DPPC/Cholesterol/DSPE-PEG(2000)NH2 liposomes (1.0/0.61/0.040), synthesised by the chosen method, and the lipid film formed is then hydrated with sterile saline NaCl 0.9% to a total lipid concentration of 10 mg/ml and dispersed by ultrasound to obtain a solution of DPPC/Cholesterol/DSPE-PEG(2000)NH2 liposomes as an intermediate for further synthesis; the liposomes obtained are then added to PVA/PLGA polymer cores not purified on Sephadex columns at a volume ratio of liposomes to polymer cores of 0.1:1 to 0.3:1, after which the whole is heated to 45 to 60° C. for 2 h to 4 h with stirring at 200 to 600 RPM, and the resulting PLGA/PVA/LIPO nanoparticles are left at 4° C. for 12 to 24 h and purified by washing twice with PBS 7 buffer.4.

5. The method according to claim. 4, characterised in that the PLGA is a 50:50 copolymer with an Mw of 24 000-38 000 g/mol.

6. The method according to claim. 4, characterised in that the PVA has a mass Mw of 30 000-70 000 g/mol.

7. The method according to claim. 4, characterised in that 0.1 M PBS buffer pH 7.4 is added to the PVA solution.

8. The method according to claim. 4, characterised in that the inner core polymer is a copolymer of poly(L-lactide-co-glycolide)—PLGA.

9. The method according to claim. 4, characterised in that the outer polymer of the core is poly(vinyl alcohol) PVA.

10. The method according to claim. 4, characterised in that the drug introduced into the core is Staurosporine in an amount of 5 to 11.5 mg.

11. The method according to claim. 4, characterised in that the drug introduced into the core is 17AAG (Tanespimycin) in an amount of 5 to 11.5 mg.

12. The method according to claim. 4, namely that Staurosporine and 17AAG (Tanespimycin) are introduced into the core at a concentration ratio of 17AAG (Tanespimycin) to Staurosporine ranging from 0.62 to 9.5

13. The method according to claim. 4, characterised in that the evaporation of dichloromethane is carried out using a magnetic stirrer at a speed of 200 to 600 RPM at a temperature of 37° C. to 40° C.

14. The method according to claim. 4, characterised in that the emulsion is obtained by agitation with an ultrasonic probe from 1 minute to 2 minutes at an amplitude of 70%-90%.

15. The method according to claim. 4, characterised in that, in order to control the quality of the manufactured PLGA/PVA polymer core, a measurement of the size of the nanoparticles is performed by means of a dynamic light scattering method.

16. The method according to claim. 4, characterised by the fact that DPPC/Cholestrol/DSPE-PEG(2000)NH2 liposomes are synthesised by means of a hydration method, preparing a solution of a lipid mixture with a concentration of: DPPC equal to 96.0 mM, Cholesterol equal to 58.8 mM, DSPE-PEG(2000)NH2 equal to 3.84 mM in chloroform.

17. The method according to claim. 16, characterised by the fact that in the hydration method the solvent is evaporated by means of a vacuum evaporator or vacuum dryer.

18. The method according to claim. 4, characterised by the fact that DPPC/Cholestrol/DSPE-PEG(2000)NH2 liposomes are synthesised by means of an ethanol injection method, preparing a solution of the lipid mixture with a concentration of: DPPC equal to 96.0 mM, Cholesterol equal to 58.8 mM, DSPE-PEG(2000)NH2 equal to 3.84 mM in ethanol.

19. The method according to claim. 18, characterised by the fact that in the ethanol injection method a solution of lipids in ethanol is injected into sterile saline mixed with a magnetic stirrer to obtain a solution of DPPC/Cholestrol/DSPE-PEG(2000)NH2 liposomes with a total lipid concentration of 10 mg/ml as an intermediate for further synthesis.

20. The method according to claim. 4, characterised in that the purification of the nanoparticles is carried out in a high-speed centrifuge at 15000 RPM for 2 to 6 min.

21. The method according to claim. 4 as follows: after each addition of PBS 7.4, the nanoparticles are dispersed using ultrasound for a period of not less than 5 seconds.

22. The method according to claim. 4 characterised by the fact that after final centrifugation of the nanoparticles a non-ionic surfactant is added to form a 40% solution in a volume ratio of 0.02:1 (surfactant:nanoparticles) or glucose.

23. The method according to claim 4 characterised in that the drug content, i.e. % EE, is determined by UV-Vis spectrophotometry for single or a mixture of drugs by dissolving PLGA/PVA/LIPO nanoparticles in DMSO with HCL according to the scheme: 25-75 uL sample, 50 uL 0.3M HCl, 25-75 uL PBS 7.4 and 850 uL DMSO, or by LC-MS method.
